SEQUENCE LISTING (11 sequences)

SEQ ID NO: 1 - protein, 1273 aa, SARS-CoV
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 222 aa, SARS-CoV
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, SARS-CoV
MYSFVSEETGTLIVNSVLLFLAFVVFLLVTLAILTALRLCAYCCNIVNVSLVKPSFYVYSRVKNLNSSRVPDLLV

SEQ ID NO: 4 - DNA, 3825 bp, SARS-CoV
atgttcgtgttcctggtcttgttgccattggtttcttctcaatgtgttaacttgaccactagaactcaattgccaccagcttacactaattctttcactagaggtgtttactaccccgacaaggtttttagatcctccgttttacattctacccaggatttgtttctgcccttcttttctaacgttacctggttccatgctatccatgtttctggtactaatggcactaagagattcgataatccagtcttgcctttcaacgatggtgtttattttgcttccaccgagaagtccaacattattagaggttggattttcggtactaccttggattctaagacccagtctttgttgatcgttaacaatgctaccaacgttgttatcaaggtttgcgagtttcaattctgcaacgatccatttttgggtgtctactaccataagaacaacaagtcttggatggaatccgagttcagagtttattcttctgctaacaactgcaccttcgaatacgtttctcaaccattcttgatggacttggaaggtaagcaaggtaacttcaagaacttgagagagttcgttttcaagaacatcgacggttacttcaagatctactctaaacataccccaatcaacttggttagagatttgccacaaggtttctctgctttggaaccattggttgatttgccaattggtatcaacatcaccagattccaaactttgttggccttgcatagatcttatttgactccaggtgattcttcttctggttggactgctggtgctgctgcttattatgttggttacttgcaacctagaaccttcctgttgaagtacaacgaaaacggtactattaccgatgctgttgattgtgctttagacccattgtctgaaactaagtgtaccttgaagtctttcaccgttgaaaagggtatctaccaaacctctaacttcagagttcaacctaccgaatctattgtcagattcccaaacattaccaacttgtgtccattcggtgaagttttcaatgctactagattcgcttctgtttacgcttggaacagaaaaagaatttccaactgcgttgctgactactctgtcttgtataattctgcttctttcagcaccttcaagtgctatggtgtttctccaactaagttgaacgatttgtgtttcaccaacgtttacgccgattcctttgttataagaggtgatgaagtcagacaaattgctccaggtcaaactggtaaaattgccgattacaattacaagttgccagatgatttcaccggttgtgttattgcttggaattctaacaacttggactctaaggttggtggcaactacaattacttgtacaggttgttccgtaagtccaacttgaagccattcgaaagagatatttccaccgaaatctatcaagctggttctactccatgtaatggtgttgaaggtttcaactgttacttcccattgcaatcttacggtttccaacctacaaacggtgttggttatcaaccatacagagttgttgttttgtccttcgaattattgcatgctccagctactgtttgtggtccaaaaaagtctaccaatctggttaagaacaagtgcgtcaatttcaacttcaacggtttgactggtactggtgttttgactgaatctaacaagaagttcttgccattccaacaattcggtagagatattgctgataccactgatgctgttagagatccacaaaccttggaaattttggatattaccccatgttctttcggtggtgtctctgttattactccaggtactaatacctctaatcaagtcgctgtgttgtaccaagatgttaactgtactgaagttccagttgctattcatgctgatcaattgactcctacttggagagtttactctaccggttctaatgttttccaaactagagctggttgtttgattggtgctgaacatgttaacaactcctacgaatgcgatattccaattggtgcaggtatttgtgcttcctaccaaactcaaactaattctccaagaagggctagatctgttgcctctcaatctattattgcttacaccatgtctttgggtgccgaaaattctgttgcttactccaacaacagtatcgctattccaactaacttcaccatttctgtcaccactgaaatcttgccagtttctatgactaagacctctgttgactgtaccatgtacatttgtggtgattctactgagtgctccaatttgttgttgcaatacggttctttctgcacccaattgaacagagctttaactggtattgctgtcgaacaagataagaacacccaagaagttttcgcccaagttaagcaaatctacaaaaccccacctatcaaggattttggtggctttaacttctcccagattttgccagatccatccaaaccatctaagaggtctttcatcgaagatctgctgttcaacaaggttactttggctgatgctggtttcatcaaacaatatggtgattgcttgggtgatattgcagccagagatttgatttgtgctcaaaagtttaatggcttgaccgttttgccaccattattgactgacgaaatgattgctcagtacacttctgctttgttagctggtactataacttccggttggacatttggtgctggtgcagctttacaaattccattcgctatgcaaatggcctacagattcaatggtattggtgttactcaaaacgtcttgtacgaaaaccagaagttgatcgccaatcaattcaactctgctatcggtaagatccaggattctttatcttctactgcttccgctttgggtaaattgcaagatgttgttaatcagaacgcccaagctttaaacaccttggttaagcaattgtcctctaatttcggtgccatctcttctgttttgaacgatatcttgtccaggttggataaggttgaagccgaagttcaaatcgatagattgattaccggtaggctgcaatccttgcaaacttatgttacccaacaattgattagggctgctgaaatcagagcttcagctaatttggctgctactaagatgtctgaatgtgtcttgggtcaatccaagagagttgatttttgcggtaagggttatcacttgatgtcttttccacaatctgctccacatggtgttgttttcttgcatgttacttatgtcccagctcaagaaaagaactttactactgctcctgctatttgccatgatggtaaagctcattttccaagagaaggtgtgtttgtctctaatggtactcattggttcgtcactcaaagaaacttttacgaaccacaaatcatcaccaccgataacacttttgtctctggtaactgtgatgttgtcatcggtattgtgaacaacactgtctatgatccattgcaaccagaattggactccttcaaagaagagttggacaagtactttaagaaccacacttctccagatgttgatttgggtgacatttctggtatcaatgcctctgttgtcaacatccaaaaagaaatcgacaggttgaacgaagtcgccaagaatttgaatgaatcgttgatcgacttgcaagagttgggcaaatatgaacagtatattaagtggccatggtacatctggttgggttttattgctggtttgattgccatcgttatggtcactataatgttgtgttgtatgacctcttgttgctcttgtttgaaaggttgttgttcctgtggttcctgctgtaaatttgatgaagatgattccgaaccagtcttgaagggtgttaagttgcattacacttgataa

SEQ ID NO: 5 - DNA, 672 bp, SARS-CoV
atggctgattccaacggtactatcaccgttgaagaactgaaaaagttgttggaacaatggaacttggtcatcggtttcttgttcttgacttggatttgcttgttgcaattcgcttacgctaacagaaacaggttcttgtacatcatcaagctgattttcttgtggttgttgtggccagttactttggcttgttttgttttggctgctgtctacagaatcaactggattactggtggtattgctattgctatggcatgtttggttggtttgatgtggttgtcttactttattgcctctttcaggttgttcgctagaactagatctatgtggtctttcaacccagagactaacatcttgttgaacgttccattgcatggtactattttgaccagacctttgttggaatctgagttggttattggtgccgttattttgagaggtcatttgagaattgctggtcatcatttgggtagatgcgatattaaggacttgccaaaagaaattaccgttgctacttccagaaccttgtcctattacaaattgggtgcttctcaaagagttgctggcgattctggttttgctgcttattcaagatacaggatcggtaactacaagttgaataccgatcactcctctagctctgataacattgctttgttggtccagtgataa

SEQ ID NO: 6 - DNA, 231 bp, SARS-CoV
atgtacagcttcgtgtctgaagaaaccggtactttgatcgttaactccgttttgttgttcttggccttcgttgttttcttgttggttactttggctattttgaccgctttgagattgtgtgcttactgctgtaatatcgtcaacgtttctttggtcaagccatccttttacgtttactccagagttaagaacctgaactcatcaagagttccagatttgttggtctgataa

SEQ ID NO: 7 - DNA, 9198 bp, SARS-CoV
cgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattccggaacggcgcgcacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccgatatcgaattcacatacaaaatgttcgtgttcctggtcttgttgccattggtttcttctcaatgtgttaacttgaccactagaactcaattgccaccagcttacactaattctttcactagaggtgtttactaccccgacaaggtttttagatcctccgttttacattctacccaggatttgtttctgcccttcttttctaacgttacctggttccatgctatccatgtttctggtactaatggcactaagagattcgataatccagtcttgcctttcaacgatggtgtttattttgcttccaccgagaagtccaacattattagaggttggattttcggtactaccttggattctaagacccagtctttgttgatcgttaacaatgctaccaacgttgttatcaaggtttgcgagtttcaattctgcaacgatccatttttgggtgtctactaccataagaacaacaagtcttggatggaatccgagttcagagtttattcttctgctaacaactgcaccttcgaatacgtttctcaaccattcttgatggacttggaaggtaagcaaggtaacttcaagaacttgagagagttcgttttcaagaacatcgacggttacttcaagatctactctaaacataccccaatcaacttggttagagatttgccacaaggtttctctgctttggaaccattggttgatttgccaattggtatcaacatcaccagattccaaactttgttggccttgcatagatcttatttgactccaggtgattcttcttctggttggactgctggtgctgctgcttattatgttggttacttgcaacctagaaccttcctgttgaagtacaacgaaaacggtactattaccgatgctgttgattgtgctttagacccattgtctgaaactaagtgtaccttgaagtctttcaccgttgaaaagggtatctaccaaacctctaacttcagagttcaacctaccgaatctattgtcagattcccaaacattaccaacttgtgtccattcggtgaagttttcaatgctactagattcgcttctgtttacgcttggaacagaaaaagaatttccaactgcgttgctgactactctgtcttgtataattctgcttctttcagcaccttcaagtgctatggtgtttctccaactaagttgaacgatttgtgtttcaccaacgtttacgccgattcctttgttataagaggtgatgaagtcagacaaattgctccaggtcaaactggtaaaattgccgattacaattacaagttgccagatgatttcaccggttgtgttattgcttggaattctaacaacttggactctaaggttggtggcaactacaattacttgtacaggttgttccgtaagtccaacttgaagccattcgaaagagatatttccaccgaaatctatcaagctggttctactccatgtaatggtgttgaaggtttcaactgttacttcccattgcaatcttacggtttccaacctacaaacggtgttggttatcaaccatacagagttgttgttttgtccttcgaattattgcatgctccagctactgtttgtggtccaaaaaagtctaccaatctggttaagaacaagtgcgtcaatttcaacttcaacggtttgactggtactggtgttttgactgaatctaacaagaagttcttgccattccaacaattcggtagagatattgctgataccactgatgctgttagagatccacaaaccttggaaattttggatattaccccatgttctttcggtggtgtctctgttattactccaggtactaatacctctaatcaagtcgctgtgttgtaccaagatgttaactgtactgaagttccagttgctattcatgctgatcaattgactcctacttggagagtttactctaccggttctaatgttttccaaactagagctggttgtttgattggtgctgaacatgttaacaactcctacgaatgcgatattccaattggtgcaggtatttgtgcttcctaccaaactcaaactaattctccaagaagggctagatctgttgcctctcaatctattattgcttacaccatgtctttgggtgccgaaaattctgttgcttactccaacaacagtatcgctattccaactaacttcaccatttctgtcaccactgaaatcttgccagtttctatgactaagacctctgttgactgtaccatgtacatttgtggtgattctactgagtgctccaatttgttgttgcaatacggttctttctgcacccaattgaacagagctttaactggtattgctgtcgaacaagataagaacacccaagaagttttcgcccaagttaagcaaatctacaaaaccccacctatcaaggattttggtggctttaacttctcccagattttgccagatccatccaaaccatctaagaggtctttcatcgaagatctgctgttcaacaaggttactttggctgatgctggtttcatcaaacaatatggtgattgcttgggtgatattgcagccagagatttgatttgtgctcaaaagtttaatggcttgaccgttttgccaccattattgactgacgaaatgattgctcagtacacttctgctttgttagctggtactataacttccggttggacatttggtgctggtgcagctttacaaattccattcgctatgcaaatggcctacagattcaatggtattggtgttactcaaaacgtcttgtacgaaaaccagaagttgatcgccaatcaattcaactctgctatcggtaagatccaggattctttatcttctactgcttccgctttgggtaaattgcaagatgttgttaatcagaacgcccaagctttaaacaccttggttaagcaattgtcctctaatttcggtgccatctcttctgttttgaacgatatcttgtccaggttggataaggttgaagccgaagttcaaatcgatagattgattaccggtaggctgcaatccttgcaaacttatgttacccaacaattgattagggctgctgaaatcagagcttcagctaatttggctgctactaagatgtctgaatgtgtcttgggtcaatccaagagagttgatttttgcggtaagggttatcacttgatgtcttttccacaatctgctccacatggtgttgttttcttgcatgttacttatgtcccagctcaagaaaagaactttactactgctcctgctatttgccatgatggtaaagctcattttccaagagaaggtgtgtttgtctctaatggtactcattggttcgtcactcaaagaaacttttacgaaccacaaatcatcaccaccgataacacttttgtctctggtaactgtgatgttgtcatcggtattgtgaacaacactgtctatgatccattgcaaccagaattggactccttcaaagaagagttggacaagtactttaagaaccacacttctccagatgttgatttgggtgacatttctggtatcaatgcctctgttgtcaacatccaaaaagaaatcgacaggttgaacgaagtcgccaagaatttgaatgaatcgttgatcgacttgcaagagttgggcaaatatgaacagtatattaagtggccatggtacatctggttgggttttattgctggtttgattgccatcgttatggtcactataatgttgtgttgtatgacctcttgttgctcttgtttgaaaggttgttgttcctgtggttcctgctgtaaatttgatgaagatgattccgaaccagtcttgaagggtgttaagttgcattacactggttcctcttcatctcatcaccatcatcatcactgataaggtacctctagagctaagcagctcgagcatcatcaccatcaccattaaagatctctgcaggatatcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaga

SEQ ID NO: 8 - DNA, 6048 bp, SARS-CoV
cgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattccggaacggcgcgcacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccgatatcgaattcggatccaagcttacatacaaaatggctgattccaacggtactatcaccgttgaagaactgaaaaagttgttggaacaatggaacttggtcatcggtttcttgttcttgacttggatttgcttgttgcaattcgcttacgctaacagaaacaggttcttgtacatcatcaagctgattttcttgtggttgttgtggccagttactttggcttgttttgttttggctgctgtctacagaatcaactggattactggtggtattgctattgctatggcatgtttggttggtttgatgtggttgtcttactttattgcctctttcaggttgttcgctagaactagatctatgtggtctttcaacccagagactaacatcttgttgaacgttccattgcatggtactattttgaccagacctttgttggaatctgagttggttattggtgccgttattttgagaggtcatttgagaattgctggtcatcatttgggtagatgcgatattaaggacttgccaaaagaaattaccgttgctacttccagaaccttgtcctattacaaattgggtgcttctcaaagagttgctggcgattctggttttgctgcttattcaagatacaggatcggtaactacaagttgaataccgatcactcctctagctctgataacattgctttgttggtccagggttcttcttcttcacatcatcaccatcaccactgataaggtacctctagactcgagcatcatcaccatcaccattaaagatctctgcaggatatcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaga

SEQ ID NO: 9 - DNA, 5607 bp, SARS-CoV
cgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattccggaacggcgcgcacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccgatatcgaattcggatccaagcttacatacaaaatgtacagcttcgtgtctgaagaaaccggtactttgatcgttaactccgttttgttgttcttggccttcgttgttttcttgttggttactttggctattttgaccgctttgagattgtgtgcttactgctgtaatatcgtcaacgtttctttggtcaagccatccttttacgtttactccagagttaagaacctgaactcatcaagagttccagatttgttggtcggttcttcttcttcacatcatcaccaccatcactgataaggtacctctagactcgagcatcatcaccatcaccattaaagatctctgcaggatatcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaga

SEQ ID NO: 10 - DNA, 10341 bp, Artificial Sequence
  Synthetic construct
cgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttagcggaagagcgcccaatacgcaaaccgcctctccccgcgcgttggccgattcattaatgcagaaatgcgcgctccggaacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccgatatcgaattcgcatgctacgtaggatccaagcttacatacaaaatgtacagcttcgtgtctgaagaaaccggtactttgatcgttaactccgttttgttgttcttggccttcgttgttttcttgttggttactttggctattttgaccgctttgagattgtgtgcttactgctgtaatatcgtcaacgtttctttggtcaagccatccttttacgtttactccagagttaagaacctgaactcatcaagagttccagatttgttggtctgataaggtacctctagactcgagcatcatcaccatcaccattaaagatctctgcaggatatcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcgcgcatcgccaaattccggaacggcgcgcacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccgatatcgaattcggatccaaacgatgttcgtgttcctggtcttgttgccattggtttcttctcaatgtgttaacttgaccactagaactcaattgccaccagcttacactaattctttcactagaggtgtttactaccccgacaaggtttttagatcctccgttttacattctacccaggatttgtttctgcccttcttttctaacgttacctggttccatgctatccatgtttctggtactaatggcactaagagattcgataatccagtcttgcctttcaacgatggtgtttattttgcttccaccgagaagtccaacattattagaggttggattttcggtactaccttggattctaagacccagtctttgttgatcgttaacaatgctaccaacgttgttatcaaggtttgcgagtttcaattctgcaacgatccatttttgggtgtctactaccataagaacaacaagtcttggatggaatccgagttcagagtttattcttctgctaacaactgcaccttcgaatacgtttctcaaccattcttgatggacttggaaggtaagcaaggtaacttcaagaacttgagagagttcgttttcaagaacatcgacggttacttcaagatctactctaaacataccccaatcaacttggttagagatttgccacaaggtttctctgctttggaaccattggttgatttgccaattggtatcaacatcaccagattccaaactttgttggccttgcatagatcttatttgactccaggtgattcttcttctggttggactgctggtgctgctgcttattatgttggttacttgcaacctagaaccttcctgttgaagtacaacgaaaacggtactattaccgatgctgttgattgtgctttagacccattgtctgaaactaagtgtaccttgaagtctttcaccgttgaaaagggtatctaccaaacctctaacttcagagttcaacctaccgaatctattgtcagattcccaaacattaccaacttgtgtccattcggtgaagttttcaatgctactagattcgcttctgtttacgcttggaacagaaaaagaatttccaactgcgttgctgactactctgtcttgtataattctgcttctttcagcaccttcaagtgctatggtgtttctccaactaagttgaacgatttgtgtttcaccaacgtttacgccgattcctttgttataagaggtgatgaagtcagacaaattgctccaggtcaaactggtaaaattgccgattacaattacaagttgccagatgatttcaccggttgtgttattgcttggaattctaacaacttggactctaaggttggtggcaactacaattacttgtacaggttgttccgtaagtccaacttgaagccattcgaaagagatatttccaccgaaatctatcaagctggttctactccatgtaatggtgttgaaggtttcaactgttacttcccattgcaatcttacggtttccaacctacaaacggtgttggttatcaaccatacagagttgttgttttgtccttcgaattattgcatgctccagctactgtttgtggtccaaaaaagtctaccaatctggttaagaacaagtgcgtcaatttcaacttcaacggtttgactggtactggtgttttgactgaatctaacaagaagttcttgccattccaacaattcggtagagatattgctgataccactgatgctgttagagatccacaaaccttggaaattttggatattaccccatgttctttcggtggtgtctctgttattactccaggtactaatacctctaatcaagtcgctgtgttgtaccaagatgttaactgtactgaagttccagttgctattcatgctgatcaattgactcctacttggagagtttactctaccggttctaatgttttccaaactagagctggttgtttgattggtgctgaacatgttaacaactcctacgaatgcgatattccaattggtgcaggtatttgtgcttcctaccaaactcaaactaattctccaagaagggctagatctgttgcctctcaatctattattgcttacaccatgtctttgggtgccgaaaattctgttgcttactccaacaacagtatcgctattccaactaacttcaccatttctgtcaccactgaaatcttgccagtttctatgactaagacctctgttgactgtaccatgtacatttgtggtgattctactgagtgctccaatttgttgttgcaatacggttctttctgcacccaattgaacagagctttaactggtattgctgtcgaacaagataagaacacccaagaagttttcgcccaagttaagcaaatctacaaaaccccacctatcaaggattttggtggctttaacttctcccagattttgccagatccatccaaaccatctaagaggtctttcatcgaagatctgctgttcaacaaggttactttggctgatgctggtttcatcaaacaatatggtgattgcttgggtgatattgcagccagagatttgatttgtgctcaaaagtttaatggcttgaccgttttgccaccattattgactgacgaaatgattgctcagtacacttctgctttgttagctggtactataacttccggttggacatttggtgctggtgcagctttacaaattccattcgctatgcaaatggcctacagattcaatggtattggtgttactcaaaacgtcttgtacgaaaaccagaagttgatcgccaatcaattcaactctgctatcggtaagatccaggattctttatcttctactgcttccgctttgggtaaattgcaagatgttgttaatcagaacgcccaagctttaaacaccttggttaagcaattgtcctctaatttcggtgccatctcttctgttttgaacgatatcttgtccaggttggataaggttgaagccgaagttcaaatcgatagattgattaccggtaggctgcaatccttgcaaacttatgttacccaacaattgattagggctgctgaaatcagagcttcagctaatttggctgctactaagatgtctgaatgtgtcttgggtcaatccaagagagttgatttttgcggtaagggttatcacttgatgtcttttccacaatctgctccacatggtgttgttttcttgcatgttacttatgtcccagctcaagaaaagaactttactactgctcctgctatttgccatgatggtaaagctcattttccaagagaaggtgtgtttgtctctaatggtactcattggttcgtcactcaaagaaacttttacgaaccacaaatcatcaccaccgataacacttttgtctctggtaactgtgatgttgtcatcggtattgtgaacaacactgtctatgatccattgcaaccagaattggactccttcaaagaagagttggacaagtactttaagaaccacacttctccagatgttgatttgggtgacatttctggtatcaatgcctctgttgtcaacatccaaaaagaaatcgacaggttgaacgaagtcgccaagaatttgaatgaatcgttgatcgacttgcaagagttgggcaaatatgaacagtatattaagtggccatggtacatctggttgggttttattgctggtttgattgccatcgttatggtcactataatgttgtgttgtatgacctcttgttgctcttgtttgaaaggttgttgttcctgtggttcctgctgtaaatttgatgaagatgattccgaaccagtcttgaagggtgttaagttgcattacacttgataaggtaccccacaattgtggggcccgggcgctagctgtacaccacaattgtggtctagagctaagcagctcgagcatcatcaccatcaccattaaagatctctgcaggatatcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaga

SEQ ID NO: 11 - DNA, 6364 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgatgcgcgcagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatgatatcctgcagagatctttaatggtgatggtgatgatgctcgagtctagaggtaccttatcactggaccaacaaagcaatgttatcagagctagaggagtgatcggtattcaacttgtagttaccgatcctgtatcttgaataagcagcaaaaccagaatcgccagcaactctttgagaagcacccaatttgtaataggacaaggttctggaagtagcaacggtaatttcttttggcaagtccttaatatcgcatctacccaaatgatgaccagcaattctcaaatgacctctcaaaataacggcaccaataaccaactcagattccaacaaaggtctggtcaaaatagtaccatgcaatggaacgttcaacaagatgttagtctctgggttgaaagaccacatagatctagttctagcgaacaacctgaaagaggcaataaagtaagacaaccacatcaaaccaaccaaacatgccatagcaatagcaataccaccagtaatccagttgattctgtagacagcagccaaaacaaaacaagccaaagtaactggccacaacaaccacaagaaaatcagcttgatgatgtacaagaacctgtttctgttagcgtaagcgaattgcaacaagcaaatccaagtcaagaacaagaaaccgatgaccaagttccattgttccaacaactttttcagttcttcaacggtgatagtaccgttggaatcagccattttgtatgtaagcttggatcctacgtagcatgcgaattcgatatcggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgttccggagcgcgcatttctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2024/0269261 A1
(43) Pub. Date: Aug. 15, 2024

(54) EXPRESSION OF SARS-COV PROTEINS, NUCLEIC ACID CONSTRUCTS, VIRUS LIKE PROTEINS (VLPS) AND METHODS RELEVANT THERETO

(71) Applicant: PREMAS BIOTECH PVT. LTD, IMT Manesar Gurgaon, Haryana (IN)

(72) Inventors: Kajal ARORA, Manesar (IN); Ruchir RASTOGI, Manesar (IN); Nupur MEHROTRA, Manesar (IN); Prabuddha KUNDU, Manesar (IN)

(73) Assignee: PREMAS BIOTECH PVT. LTD, IMT Manesar Gurgaon, Haryana (IN)

(21) Appl. No.: 17/908,854

(22) PCT Filed: Mar. 4, 2021
(86) PCT No.: PCT/IB2021/051828

§ 371 Date: Sep. 1, 2022

(30) Foreign Application Priority Data
Mar. 4, 2020 (IN) 202011009383

(51) Int. Cl. A61K 39/215; A61K 39/00; A61P 31/14; C07K 14/005; C12N 1/16; C12N 7/00; C12N 15/81; C12R 1/865

(52) U.S. Cl. CPC A61K 39/215; A61P 31/14; C07K 14/005; C12N 1/165; C12N 7/00; C12N 15/81; A61K 2039/5258; C12N 2770/20022; C12N 2770/20023; C12N 2770/20034; C12R 2001/865

(57) ABSTRACT

The present invention relates to expression of SARS-CoV like virus proteins [S, M and E] proteins; recombinant polynucleotides, polypeptides; constructs, virus-like particles (VLPs); immunogenic compositions or vaccines comprising Virus Like Particles (VLPs). Method of producing the VLPs/expressing the multi-subunit virus like proteins and method for co-expression of multi-subunit and virus like proteins (VLPs) are also provided. The present invention also provides strategies, methods, systems, kits and combinations for scalable expression, purification and enhanced production of the virus like proteins of SARS-CoV while maintaining their size range and composition. Such multi-subunit VLPs can be utilized to make immunogenic compositions or vaccines.

### FIELD OF INVENTION

The present invention broadly lies in the field of virology and viral vaccines. More particularly, the present invention relates to expression of SARS-CoV like virus proteins; recombinant polynucleotides, polypeptides; virus-like particles; immunogenic compositions or vaccines comprising virus like particles and methods of producing and purifying the same.

### BACKGROUND OF THE INVENTION

Virus-like particles (VLPs) resemble their corresponding native viruses with similar overall structure but lessened original infectious ability due to the absence of viral genome. VLPs are symmetrically built from hundreds of coat proteins, which can be genetically engineered to present a regular arrangement of epitope chains on the desired positions of the outer surface.

Currently, VLPs have been widely applied for a variety of applications such as vaccines, antibody development, delivery systems, bioimaging, and cell targeting. More precisely, VLPs have proven to be promising candidate vaccines since they: (i) do not comprise a nucleocapsid and are non-infectious and therefore safe to produce and use, (ii) are more immunogenic than subunit vaccines because they provide the necessary spatial structure for display of epitopes, and (iii) elicit humoral, cell-mediated and importantly, mucosal immunity (Krueger et al., Biol. Chem., 380:275-276, 1998).

WO2017/040387A2 discloses a virus-like particles (VLPs) comprising an antigenic RSV protein and a composition comprising these VLPs, as well as methods for making and using these VLPs.

U.S. Pat. No. 8,592,197 B2 discloses a macromolecular protein structure containing (a) a first influenza virus M1 protein and (b) an additional structural protein, which may include a second or more influenza virus M1 protein; a first, second or 50 more influenza virus HA protein; a first, second, or more influenza virus NA protein; and a first, second, or more influenza virus M2 protein.

US 2010/0120092 A1 discloses chimeric or recombinant virus like particles comprising (i) S polypeptide of an avian hep adenovirus and (ii) a chimeric fusion protein comprising a polypeptide of interest Covalently attached to a particle-associating portion of L polypeptide of an avian hepadnavirus.

Of the various biological molecules being developed as potential vaccines or therapeutics, development of various types of VLPs as next generation vaccines has accelerated over the past two decades. However, it has been observed that the expression levels of viral proteins in different platforms vary considerably. In general, the secretory expression of glycoproteins is difficult. Budding from cell membrane to get envelop is a key step during the eVLP (enveloped virus like particle) formation process. If eVLP is not efficiently secreted, a cell lysis or other extraction step might be required, and these steps increase the difficulty for further purification. A common way to improve expression level of transmembrane glycoproteins is to delete or replace the transmembrane region which anchors the protein in the membrane (Wang et al, 2018).

Eukaryotic expression systems are more suitable for the production of eVLP. Yeast expression systems, especially those based on Saccharomyces cerevisiae and Pichia pastoris, have advantages such as scalable fermentation, low production costs, and PTM process. Yeast has advantages such as scalable fermentation, low risk of contamination by adventitious agents, low production costs and the ability to produce VLPs with reliable qualities. Accordingly, yeast platform has been a preferable choice for productions of both, the nonenveloped viruses but also of enveloped viruses. It is also understood that the quality and quantity of yeast derived VLPs is largely influenced by the choice of plasmid and promoter, and the ratio of the structural proteins produced. VLPs derived from nonenveloped or enveloped viruses can broadly be classified as the single-layered and multi-layered VLPs (Letters in Applied Microbiology, 2016).

The appropriate formation of complex multi-layered VLPs is restricted to a narrow range of association energies and protein concentrations. Thus the choice of the host system and design of expression is critical for a successful assembly. In literature, there are types of plasmids/vectors used in the transformation of yeast cell like Autonomously Replicating Plasmids (ARS), Yeast centromere Plasmids (YCps), Yeast Integrating Plasmids (YIp), Episomal Vectors (YEP plasmids) and Yeast Linear Plasmids (YLp). Episomal expression vectors (Yep) have been used in literature for the production of VLPs which utilises multiple episomal vectors. This may cause presence of excess foreign DNA and load in the cells due to presence of these plasmids as multiple copies.

Two licensed vaccines, the HBV vaccine Engerix-B® and HPV vaccine Gardasil® have been manufactured using this system. However, in the case of HIV-2, Saccharomyces cerevisiae cells fail to support the multimerization of the enveloped Gag protein into VLPs and particle budding from the membrane.

Several other routes for expression of antigens in multi-layered VLPs have also been described in literature. As one of the strategies used, two antigens VP1 and VP2 for Parvovirus VLP have been co-expressed under a bicistronic plasmid under the control of the same promoter (ADH2/GAPDH). In this case, it is seen that expression of VP2 antigen is lower than VP1, indicating that it is critical to maintain the ratio of both the expression constructs.

In another example, VLP for Coxsackievirus A16 and Enterovirus 71, which are Picornaviridae and are major causative agents of hand, foot and mouth diseases (HFMD) (Zou et al. 2012), have been produced. These are composed of VP0, VP1 and VP3 antigens and are each processed from P1 polypeptide by the 3CD viral protease (Li et al. 2013; Zhao et al. 2013a). Multilayered VLPs of coxsackievirus A16 and enterovirus 71 have been produced in both S. cerevisiae and P. pastoris by intracellular expression strategies (Li et al. 2013; Zhao et al. 2013a; Zhang et al. 2015; Wang et al. 2016). There are two different types of expression strategies for expression plasmids. One is to use two separate expression plasmids, one for producing the P1 region and the other for the 3CD protease (Zhao et al. 2013a), and the other strategy is to use a single expression plasmid for both regions (Zhang et al. 2015; Zhou et al. 2016). In both cases, the VLPs produced had potential as vaccines but Zhang et al. obtained significantly higher levels of expression of enterovirus 71 antigens from cells transformed with the single expression plasmid than from those transformed with the two separate plasmids.

In another example, Rotavirus VLPs which are composed of VP2, VP6 and VP7 have shown to be produced intracellularly in S. cerevisiae (Rodriguez-Limas et al. 2011), wherein a single plasmid harbours the VP2, VP6 and VP7 genes was more effective than using three individual plasmids, because the introduction of three individual plasmids creates a metabolic burden and reduced growth rate of the yeast. Moreover, it is important to control the VP7:VP6 expression ratio during the production of rotavirus structural proteins in order to improve the yield of authentic multilayered VLPs.

On another aspect, stability of the VLP-based vaccines is one of the most significant and challenging issues. VLPs being multimeric structures are generally more stable than subunit vaccines, however, the lack of the viral genome makes them unstable when the conditions change, especially during downstream processing (DSP) and purification. Generally, it is seen that eVLPs having a host-derived envelope are more sensitive to the external environment than the protein-only VLPs. Variations in conditions, e.g., temperature, shear force and chemical treatment can destroy the integrity and stability of the particles, this structural destruction further leads to the reduction in immunogenicity of eVLPs, thus, robust purifications processes are required so as to maintain the VLP structure, conformation and immunogenicity

With the outbreak of severe acute respiratory syndrome (SARS), there is an urgent need for the development of vaccines for preventing SARS caused by Coronavirus (SARS-CoV). Coronaviruses commonly cause infections in both humans and animals. Coronavirus virus particles contain four main structural proteins. These are the spike (S), membrane (M), envelope (E), and nucleocapsid (N) proteins, all of which are encoded within the 3′ end of the viral genome.

The S protein (˜150 kDa), homotrimers of the virus encoded S protein make up the distinctive spike structure on the surface of the virus. S is cleaved by a host cell furin-like protease into two separate polypeptides noted S1 and S2. S1 makes up the large receptor-binding domain of the S protein while S2 forms the stalk of the spike molecule. Interactions of the spike protein from the SARS family of viruses through their receptor binding domain (RBD)-CoV spike protein receptor-binding domain (RBD) and host receptor angiotensin-converting enzyme 2 (ACE2) has been shown responsible for both cross-species and human-to-human transmissions of virus. Thus, using S protein or its domains as immunogens may provide protective response against the spread of the virus and also aid in its clearance.

However, Protein attributes of S protein showed the protein has extremely high number of cysteine residues along with high hydrophobicity which describes tendency of protein towards insoluble expression or aggregations if expressed in prokaryote system. Protein is very big in size and also has high proline residues a structure breaker may increase the instability of the protein, which makes its expression and purification difficult for vaccine and other purposes.

The M protein is the most abundant structural protein in the virion. It is a small (˜25-30 kDa) protein with 3 transmembrane domains and is thought to give the virion its shape. Its interaction with the S protein and N protein is essential for viral assembly and budding. The N terminal and the C terminal of the protein have been found to be highly immunogenic and have shown to induce antibody responses in hosts infected by coronavirus or immunized by attenuated recombinant virus expressing the M Protein. Moreover, the M proteins of coronaviruses contain highly conserved glycosylation sequences, and their glycosylation may be related to the interaction between virus and host. In Alpha coronaviruses, it has been demonstrated that M protein cooperates with the Spike during the cell attachment and entry. Therefore, mutations occurring at the N-terminus region, which is exposed to the virus surface, could play a key role in the host cell interaction Thus, immunizing with the protein can help generate neutralizing antibodies which can help clear the virus

The E protein (˜8-12 kDa) transmembrane protein is found in small quantities within the virion. The E protein facilitates assembly and release of the virus and also has other functions. E proteins play a part in viral assembly and morphogenesis and blocking its function has been helpful to contain the virus. The E protein is conserved across β-coronaviruses (Bianchi et al., 2020; Biomed Research International, 2020) E protein, as a pentameric viroporin-like protein, is a minor component of the virus membrane though it is deemed to be important for many stages of virus infection and replication. It is observed that E protein attributes shows that the protein is high cysteine residues with extremely high hydrophobicity which describes their propensity towards insoluble expression if expressed in prokaryotic host. The observed parameter keeps the protein in difficult to express category and suggest the suitability of eukaryotic host system for expression being membrane protein.

Studies have shown that novel coronaviruses can escape the host immune response either by exposing non-neutralizing epitopes on their RBDs or due to emerging mutations in the SARS-CoV-2 S sequence which mediate escape from neutralizing antibody responses induced by immunogens designed from the SARS-CoV2. With most of the vaccines targeting the S protein, emergence of antibody-resistant SARS-CoV-2 variants might limit the therapeutic usefulness of these vaccines.

One possible route to mitigate this resistance to recognition owing to mutations can be the use of multiple antigens for generating antibodies and allowing the immune system to recognise multiple epitopes form multiple antigens. As such, other proteins of SARS-CoV-2 may also play important roles while developing suitable vaccine candidates. Thus, the presence of three surface proteins of the SARS-CoV2 “S”, “E” and “M” provide a wider repertoire of antigens for an effective antibody mediated immune response and vaccines serve as one of the most important therapeutic mechanisms which help to get acquired immunity against a particular disease.

Precisely, generation of a humoral immune response is central to development of a vaccine. The antibodies play a significant role in preventing viral infection by either acting as neutralizing, enhancing phagocytosis by immune cells and agglutination. However, during mass vaccination campaigns, it is envisaged that a large number of doses shall be administered over a short period. There is a high probability of coincidental adverse events.

In such an event, to demonstrate safety of a candidate across multiple dose ranges is of utmost importance, it is also required to demonstrate stringent safety checks of the vaccine candidate during pre-licensure stage. Also, efficient expression systems or platforms capable of expressing SARS-CoV virus like proteins are lacking, which hinders research and development in this area.

The present invention aims to obviate the problems in prior art and endeavors to provide efficient VLPs along with their purification and method for expression of SARS-CoV proteins and methods applicable thereto. The present invention also provides an immunogenic composition and vaccine comprising the VLPs obtained from the S, E and M proteins of SARS-CoV.

### OBJECTIVE OF THE INVENTION

An important objective of the present invention is to provide recombinant VLPs of SARS-CoV and their utilization as a vaccine candidate.

Another important objective of the present invention is to produce recombinant multi-subunit VLPs comprising different proteins from SARS-CoV.

Another important objective of the present invention is to provide method of producing the desired multi-subunit VLPs.

Yet another objective of the present invention is to develop an efficient method for co-expression of multi-subunit and virus like proteins from SARS-CoV, such as, but not limited to S, M and E proteins.

Another important objective of the present invention is to provide related recombinant polypeptides and recombinant polynucleotides.

Still another objective is to provide strategies, methods, systems, kits and combinations for consistent scalable expression and enhanced production of the VLPs of SARS-CoV which maintains its size range and composition.

Still another objective is to provide a method of producing the scalable amount of VLPs.

Still another objective is to provide a method of purification of VLPs in high yields.

Yet another objective of the present invention is to provide an immunogenic composition comprising VLPs of SARS-CoV.

Still another objective is to provide safe and efficacious vaccines comprising recombinant VLPs) of SARS-CoV.

### BRIEF DESCRIPTION OF FIGURES AND DRAWINGS

The accompanying drawings illustrate some of the embodiments of the present invention and, together with the description, explain the invention. These drawings have been provided by way of illustration and not by way of limitation.

FIG. 1: (A) Outline restriction map for S protein expression in episomal vector (B) Immunoblot analysis of expressed S protein at small scale (C) Immunoblot analysis of expressed S protein in microsomal fraction as resolved on a 6% gel (loaded as 20 μl and 40 μl) (D) Peptide mapping analysis of expressed His tagged S protein. The green highlighted sequence was identified in high confidence for S protein in peptide mapping analysis.

FIG. 2: (A) Restriction map for M protein expression in episomal vector (B) Immunoblot analysis of expressed M protein at small scale (C) Immunoblot analysis of expressed M-His tag protein in microsomal fraction (loaded as 10 μl, 20 μl and 40 μl) (D) Peptide mapping analysis of expressed M His Tag protein in microsomal fraction. The green highlighted sequences were identified in high confidence for M protein in peptide mapping analysis.

FIG. 3: (A) Restriction map for E protein expression in episomal vector (B) Immunoblot analysis of expressed E_His tag protein at small scale, (C) Immunoblot analysis of expressed E-His tag protein in microsomal fraction (loaded as: 10 μl, 20 μl, 40 μl). (D) Peptide mapping analysis of expressed E His Tag protein in microsomal fraction. The green highlighted sequences were identified in high confidence for E protein in peptide mapping analysis.

FIG. 4: Immunoblot confirmation of presence of all three antigens S (FIG. 4A), E (FIG. 4B) and M (FIG. 4C) in the VLP preparation. Samples were prepared post-harvest of the culture as per the established protocols.

FIG. 5: Peptide mapping analysis of the VLP sample was performed using the Mass spectrometry using a standard protocol. All three antigens have been confirmed for their presence in the formulation in this analysis.

FIG. 6: Antigen specific IgG response to S, E and M proteins as measured by ELISA in sera samples from mice immunized with eVLP alone or with adjuvant (Alhydrogel+eVLP). Error bars indicate SEM.

FIG. 7: ELISA results showing eVLP specific IgG antibody titers in BALB/c mice immunized with indicated doses of eVLP either with or without AH.

FIG. 8: Neutralization potential (NP) of sera from immunized mice relative to neutralization potential of convalescent patients' as reference.

FIG. 9: PRNT50 titers of sera samples from different immunized mice groups

FIG. 10A: BrDU cell proliferation studies of PBMCs in patient samples subjected to two doses of VLP formulation (2.5 μg-2 Fold; 5 μg: 1.45 Fold) in comparison to PBMCs from healthy individuals.

FIG. 10B: Cell proliferation studies of PBMCs shows elevated levels of IFN-γ in the VLP stimulated patients PBMCs as compared to healthy controls.

FIG. 11: Stoichiometric ratio calculation for S, M and E in the produced VLP from co expression. Standard curve for S1 protein (B) Standard curve for M protein (C) E protein standard curves.

### SUMMARY OF THE INVENTION

The present invention relates to expression of SARS-CoV like virus proteins; recombinant polynucleotides, polypeptides; constructs, virus-like particles; immunogenic compositions or vaccines comprising virus like particles. Method of producing the VLPs/expressing the multi-subunit virus like proteins. The present invention also provides an efficient method for co-expression of multi-subunit and virus like proteins such as SARS-CoV S, M and E proteins and related recombinant polypeptides and recombinant polynucleotides.

The present invention also provides immunogenic compositions or vaccines comprising the VLPs of SARS-CoV. multi-subunit VLPs can be utilized to make.

The present invention also provides strategies, methods, systems, kits and combinations for scalable expression, purification and enhanced production of the virus like proteins of SARS-CoV while maintaining their size range and composition.

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### Definitions

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive.

The term “expression vectors” defines a plasmid or virus designed for gene expression in cells.

The term “host cell” means a host cell used for generation of recombinant proteins.

The term “viral proteins” includes proteins generated by viruses including enzyme proteins as well as structural proteins such as capsid and viral envelope.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

Now vis-à-vis the present invention, an important embodiment of the present invention is to provide recombinant VLPs of SARS-CoV and their utilization as a vaccine candidate.

Another important embodiment of the present invention relates to production of recombinant multi-subunit VLPs comprising different proteins from SARS-CoV such as but not limited to, “S” Spike; “M” Membrane protein and “E” protein derive from SARS-CoV-2 virus which have high importance in vaccine development.

Another embodiment of the present invention is to provide method of producing the desired multi-subunit VLPs.

Yet another embodiment of the present invention pertains to an efficient method for co-expression of multi-subunit and virus like proteins from SARS-CoV, such as, but not limited to S, M and E proteins.

Another important embodiment of the present invention is to provide recombinant polypeptides and recombinant polynucleotides of S, M and E proteins from SARS-CoV.

Still another embodiment of the present invention is to provide strategies, methods, systems, kits and combinations for scalable expression, purification and enhanced production of the VLPs of SARS-CoV.

Still another embodiment of the present invention is to provide immunogenic compositions comprising the VLPs of SARS-CoV.

Still another important embodiment of the present invention is to provide vaccines comprising recombinant VLPs of SARS-CoV.

In an important aspect of the present invention, codon optimization was done in respective genes to obtain modified S, M and E proteins with increase in de novo mRNA synthesis rate and stable mRNA production for efficient expression of proteins and VLPs with high yields and efficacy. The VLPs of the present invention containing the recombinant proteins able to generate high immune response and are ideal to be used in an immunogenic composition or vaccine.

In an embodiment, present invention provides a recombinant polypeptide selected from the group comprising the SEQ ID Nos 1, 2 and 3 and variants thereof, wherein the recombinant polypeptide belongs to proteins selected from Spike (S), Membrane (M) and Envelope (E) proteins, respectively, from SARS-CoV family, and wherein the said proteins are either individually expressed or co-expressed.

In another embodiment, wherein said variant of the polypeptides comprises a sequence having at least 70% sequence identity to the polypeptide sequence selected from the group comprising SEQ ID Nos 1, 2 and 3.

In still embodiment, wherein said polypeptides are obtained after codon optimization.

In yet another embodiment, said S protein is obtained after individual expression of SEQ ID NO 1 or a variant thereof.

In still another embodiment, said recombinant polypeptide is a full-length S protein which is a single pass type I membrane glycoprotein.

In yet another embodiment, said M protein is obtained after individual expression of SEQ ID NO 2 or a variant thereof.

In still another embodiment, wherein said recombinant polypeptide is a full-length M protein which is a multi-pass membrane glycoprotein.

In another embodiment, said E protein is obtained after individual expression of SEQ ID NO3 or a variant thereof.

In yet another embodiment, said recombinant polypeptide is a full-length E protein which is a single pass type III membrane glycoprotein.

In another embodiment, the present invention provides a recombinant molecule comprising recombinant polypeptides wherein said molecule is a complex formed by assembly of polypeptides defined in SEQ ID NO 1, 2 and 3 or variants thereof and is obtained after co-expression of S, M and E proteins to form an ordered aggregate.

In another embodiment, said recombinant molecule assembles to form VLPs.

In another embodiment, wherein said, the present invention provides a recombinant polynucleotide selected from the group comprising the SEQ ID Nos 4, 5 and 6 or variants thereof, wherein the polynucleotides are selected from Spike (S), Membrane (M) and Envelope (E) genes from SARS-CoV family, and wherein the said genes are cloned individually or in combination.

In yet another embodiment, wherein said variant of the nucleotides comprises a sequence having at least 60% sequence identity to the polynucleotide sequence selected from the group comprising SEQ ID NOs 4, 5 and 6.

In still another embodiment, said polynucleotides are codon biased and optimized.

In yet another embodiment, said S gene has SEQ ID NO 4 and variants thereof.

In another embodiment, said M gene has SEQ ID NO 5 and variants thereof.

In still another embodiment, said E gene has SEQ ID NO 6 and variants thereof.

In another embodiment, said recombinant polynucleotides as claimed in claim 9, wherein the polypeptides or polynucleotides are selected from, but not limited to, Spike (S), Membrane (M) and Envelope (E) proteins from SARS-CoV-2.

In another embodiment, the present invention provides a method of expressing SARS-CoV proteins comprising the steps of:
i. selecting the target genes from the group comprising of S, E and M proteins;
optimizing and codon biasing of target gene sequences; ii.
iii. preparing the construct comprising target genes and yeast-based expression vectors by cloning of target genes individually or in combination to co-express the proteins into the expression vector, wherein the expression vector is yeast based expression vector selected from episomal expression vector and/or integrative expression vector or a combination of both;
iv. transforming the constructs into Protease deficient yeast host cell to obtain target proteins.

In another embodiment, the present invention provides a method of expressing S protein from SARS-CoV, wherein the method comprises the steps of:
i. selecting the target gene S protein;
ii. optimizing and codon biasing of target gene sequence;
iii. preparing the construct comprising S gene and yeast-based expression vector by cloning of S gene individually to express the said protein into the expression vector, wherein the expression vector is yeast based episomal or integrative expression vector;
iv. transforming the construct into Protease deficient yeast host cell to obtain target protein.

In yet another embodiment, the present invention provides a method of expressing M protein from SARS-CoV, wherein the method comprises the steps of:
i. selecting the target gene M protein;
ii. optimizing and codon biasing of target gene sequence;
iii. preparing the construct comprising M gene and yeast-based expression vector by cloning of M gene individually to express the said protein into the expression vector, wherein the expression vector is yeast based integrative or episomal expression vector;
iv. transforming the construct into Protease deficient yeast host cell to obtain target protein.

In another embodiment, the present invention provides a method of expressing E protein from SARS-CoV, wherein the method comprises the steps of:
i. selecting the target gene E protein;
ii. optimizing and codon biasing of target gene sequence;
iii. preparing the construct comprising E gene and yeast-based expression vector by cloning of E gene individually to express the said protein into the expression vector, wherein the expression vector is yeast based episomal or integrative expression vector;
iv. transforming the construct into Protease deficient yeast host cell to obtain target protein.

In another embodiment, the present invention provides a method of expressing the SARS-CoV proteins wherein the method of co-expressing S, E and M proteins comprises the steps of:
i. selecting the S, E and M proteins from SARS-CoV;
ii. cloning “S” and “E” proteins into an episomal expression vector to obtain a construct having SEQ ID NO 10 and “M” protein into integrative expression vector to obtain a construct having SEQ ID NO 11;
iii. optimizing and codon biasing of target gene sequences;
iv. preparing the construct comprising the selected genes and yeast vectors;
v. transforming the constructs into yeast host;
vi. over-expressing the target multi-subunit proteins in the cell.

In another embodiment, the present invention provides a method wherein all the three proteins and variants thereof are either co-expressed individually as episomal or integrative or combinations thereof.

In still another embodiment, the proteins are selected, but not limited to, the S, M and E Corona virus proteins.

In yet another embodiment, said yeast host is Saccharomyces cerevisiae.

In another embodiment, the present invention provides a construct comprising the target genes selected from “S”, “M” and “E” proteins of SARS-CoV having SEQ ID Nos 7, 8 and 9 or variants thereof, individually or in combination to co-express in yeast-based expression vectors, wherein the vectors are selected from episomal and/or integrative vectors and wherein any two proteins are expressed in episomal and one protein is expressed in integrative expression vector.

In another embodiment, said construct comprises S protein having SEQ ID NO 1 of SARS CoV virus and a yeast episomal expression vector.

In another embodiment, said construct comprises M protein having SEQ ID NO 2 of SARS CoV virus and a yeast integrative or episomal expression vector or integrative expression vector.

In another embodiment, said construct comprises E protein having SEQ ID NO 3 of SARS CoV virus and a yeast episomal or integrative expression vector.

In another embodiment, said construct is a multiprotein construct comprising “S” and “E” proteins expressed into an episomal expression vector to obtain a construct having SEQ ID NO 10 and “M” protein expressed into integrative expression vector to obtain a construct having SEQ ID NO 11.

In another embodiment, the present invention provides a recombinant Corona virus like particle (VLP) comprising SARS-CoV proteins, wherein the SARS-CoV proteins are selected from;
(i) a SARS-CoV-virus Spike proteins (S);
(ii) a SARS-CoV-virus Membrane proteins (M); and
(iii) a SARS-CoV-virus Envelope proteins (E),
   or variants thereof.

In another embodiment, said VLPs comprise one or more SEQ IDs selected from 1, 2 and 3 encoding S, M and E proteins or their variants thereof, in a yeast cell under conditions which permit the formation of VLPs.

In another embodiment, the present invention provides a method said VLPs maintain a size range and stoichiometry range for all the co-expressed proteins.

In another embodiment, the present invention provides a method of preparing the target VLPs comprising the target proteins selected from S, M and E proteins of SARS-CoV, individually or in combination, comprising the steps of:
i. inserting the target gene selected from S, M and E proteins of SARS-CoV into protease deficient yeast host cell by either episomal construct or integrative construct;
ii. transforming the selected genes in protease deficient yeast host strain;
iii. selecting the transformants on selective Yeast Nitrogen Base (YNB) Glucose medium and without LEU auxotrophic marker in case of integrative construct and without URA auxotrophic marker in case of episomal construct;
iv. transforming the selected genes in protease deficient yeast host strain;
v. selecting the transformants on YNB with Glucose without URA and LEU auxotrophic marker plates;
vi. performing transformation;
   wherein, transformation is a sequential transformation and performed using Lithium acetate/SS-DNA/PEG mediated protocol.

In another embodiment, the present invention provides a method of preparing the target VLPs, comprising the target M protein of SARS-CoV, individually, comprising the steps of:
i. Integrating/episomally inserting the M gene into protease deficient yeast host cell;
ii. transforming the M gene in Protease deficient yeast host strain;
iii. selecting the transformants on Yeast Nitrogen Base (YNB) Glucose medium without LEU auxotrophic marker in case of integrative construct and without URA auxotrophic marker in case of episomal construct;
iv. performing transformation;
   wherein, transformation is performed using Lithium acetate/SS-DNA/PEG mediated protocol.

In another embodiment, the present invention provides a method of preparing the target VLPs comprising the target E and M protein of SARS-CoV, in combination, comprising the steps of:
i. Integrating/episomally inserting the E or M gene into protease deficient yeast host cell;
ii. transforming the E or M gene in Protease deficient yeast host strain
iii. selecting the transformants on Yeast Nitrogen Base (YNB) Glucose medium without LEU auxotrophic marker in case of integrative construct and without URA auxotrophic marker in case of episomal construct;
iv. transforming the E or M gene in Protease deficient yeast host strain for co expression;
v. selecting the transformants on YNB Glucose without URA and LEU auxotrophic marker plates for co-expression using episomal and integration vector; and selecting the transformants on YNB Glucose without URA for expression of both using episomal vector or YNB Glucose without LEU for expression of both using integration vector;
vi. performing transformation;
   wherein transformation is performed using Lithium acetate/SS-DNA/PEG mediated protocol.

In another embodiment, the present invention provides a method comprising the target proteins selected from S, M and E proteins of SARS-CoV, in combination, comprising the steps of:
i. inserting the target gene selected from S, M and E proteins of SARS-CoV into protease deficient yeast host cells;
ii. selecting the transformants on selective Yeast Nitrogen Base (YNB) Glucose medium and without LEU auxotrophic marker;
iii. transforming the selected genes in protease deficient yeast host strain;
iv. selecting the transformants on YNB with Glucose without URA and LEU auxotrophic marker plates;
v. performing transformation;
   wherein the transformation is a sequential transformation and is performed using Lithium acetate/SS-DNA/PEG mediated protocol by incubating the plates at 28° C. for 2-4 days for S, E, and M proteins and cultures were grown in YNB Glucose without URA and LEU media at 28° C. for 36 hr and control with URA media.

In another embodiment, the present invention provides a method of producing the scalable amount of VLPs comprising the steps of:
i. overexpressing recombinant proteins or VLPs in a transformed yeast host cell culture in a batch or fed batch cultivated system using high density culture, wherein the media is supplemented with additives, such as glucose, glycerol, either at the time of inoculation or later at the time when the cells are grown to a high cell density, wherein high cell density is a cell density where the transformed yeast cells are grown to a density of up to 60 at OD600 and up to 120-300 g/L, WCW in a time interval of 12-24 hours at a growth temperature of 28-32° C.;
ii. growing the transformed yeast cell in culture to a stage of log phase growth;
iii. cooling the cultivation media to a temperature in range of 25-28° C.;
iv. inducing the cultivation media with induction agents;
v. harvesting the yeast cells containing the VLP by centrifugation or a microfiltration step, or a mixture of both;
vi. washing the harvested yeast cells with cell lysis buffer consisting of a diafiltration or buffer exchange step or an ultrafiltration step, or a mixture of both and resuspending the yeast host cells in the lysis buffer;
vii. disrupting the yeast host cells in lysis buffer by mechanical force or ultrasonic waves, or a French press or a method of cell lysis, or a combination of the above;
viii. purifying the VLPs obtained in step (vi), using an ion exchange resin or a mix mode resin or a combination of the two wherein said exchange resin is an anion or cation or mix mode resin, Capto Core 70;
ix. eluting the VLPs with a buffer containing up to 1-2 M KCl;
x. dialysing the VLPs with a formulation buffer comprising 20-100 mM Potassium phosphate pH 7.2.
xi. supplementing the VLPs with 50-100 mM KCl, optionally along with 0.0005-0.001% Tween 80 and 2-10% Sucrose.
xii. filter-sterilizing the dialyzed VLPs using 0.2 μm filter;
xiii. determining the yield of purified VLP.

In still another embodiment, the high density culture of yeast cells and cultivation medium is supplemented with an amount of an induction agent such as galactose, glycerol or a mixture of both wherein the period of induction is from 48-120 hours at 25-28° C.

In another embodiment, the cultivation medium is further supplemented with a boosting solution containing supplements selected from peptides, amino acids, tryptone and yeast extract.

In another embodiment, the cultivation media is saturated by air to a level 20-80% saturation.

In another embodiment, the lysis buffer contains 20 mM-100 mM Potassium phosphate pH 7.2, 0.0005%-0.001% Tween 80 or Tween 20 or a non-ionic detergent, 2 mM PMSF and wherein the lysis buffer is optionally supplemented with a nuclease.

In another embodiment, the yeast cells containing the VLP in lysis buffer is disrupted by mechanical force, specifically with a high pressure ultrasonic waves, or a method of cell lysis, where in the yeast cells are lysed or broken to obtain a lysed medium of 60-100% lysed cells in the lysis buffer.

In another embodiment, said VLPs are harvested by centrifugation step, or a diafiltration or an ultrafiltration step microfiltration step, or a mixture of both, to produce partially isolated VLPs.

In another embodiment, the eluted VLPs are dialysed by buffer exchange with a formulation buffer, consisting of 20-100 mM Potassium phosphate pH 7.2.

In another embodiment, the present invention provides a method an immunogenic composition, comprising the VLPs, along with pharmaceutically acceptable excipients, adjuvants and/or stabilizers, wherein the VLP formulation is administered with or without an adjuvant.

In another embodiment, said composition is administered intranasal, mucosal, intradermal, subcutaneous, intramuscular, sublingual or oral.

In another embodiment, the present invention provides a vaccine comprising the VLPs, wherein said vaccine induces an immune response in a subject.

In another embodiment, said immunogenic composition or the vaccine, wherein said immunogenic composition or the vaccine induces the immune response in a subject against multiple serotypes or clades of SARS-CoV virus.

In another embodiment, said SARS-CoV virus is SARS-CoV2 virus.

In another embodiment, the subject is a human and/or animal.

In present invention both integration and episomal expression vector are simultaneously used for co-expression of full-length SARS CoV-2 structural glycoprotein antigens Spike, Membrane and Envelope for the VLP production using S. cerevisiae host system. The designed strategy for the heterologous co-expression of three structural proteins includes the integration of capsid Membrane (M) protein into the protease deficient yeast host genome while the Spike (S) and Envelope (E) proteins have been cloned and co expressed in episomal vector to reduce the excess foreign DNA interference and load in cell for the enhanced efficient expression of all the proteins in desired stoichiometry ration response.

Electron microscopy of the SARS-CoV-2 virus has, shown it to be pleomorphic with a size range distribution from 52 to 200 nm. Further structural analysis for protein spacing data, basis the size from coronaviruses have shown a 4 to 5 nm distance between the M dimer molecules. Basis the size, this leads to ˜1100 molecules of M2 in a SARS-CoV virus. Further, the model predicted presence of ˜90 spikes per particle which exists as a trimer of size 9 to 12 nm and gives a corona appearance to the virus. Further study from TGEV corona viruses showed that 15 to 30 copies of E protein are present per 100 to 200 molecules of S protein.

Thus, in the current invention, a new approach of co-expression of three proteins is demonstrated from three independent cassettes which assemble together to form a VLP. While the expression of the “M” protein was driven from a genomic integration done using pYRI100, the co-expression of “S” and “E” was driven by an episomal vector pYRE100. This strategy has led to successful expression of SARS-CoV-2 antigens which lead to formation of VLP at high concentration in the yeast cytoplasm.

Present invention also shows appropriate assembly of the Corona like structure and ratio of three recombinantly co-expressed structural proteins S, M and E in produced stable VLPs using S. cerevisiae as host system and successfully used in eliciting the immune response against all three proteins in mice and Nabs.

### EXAMPLES

The present invention is further described herein below by way of illustration and more particularly, the following paragraphs are provided in order to describe the best mode of working the invention and nothing in this section should be taken as a limitation of the claims.

### Example 1: Expression of S Protein

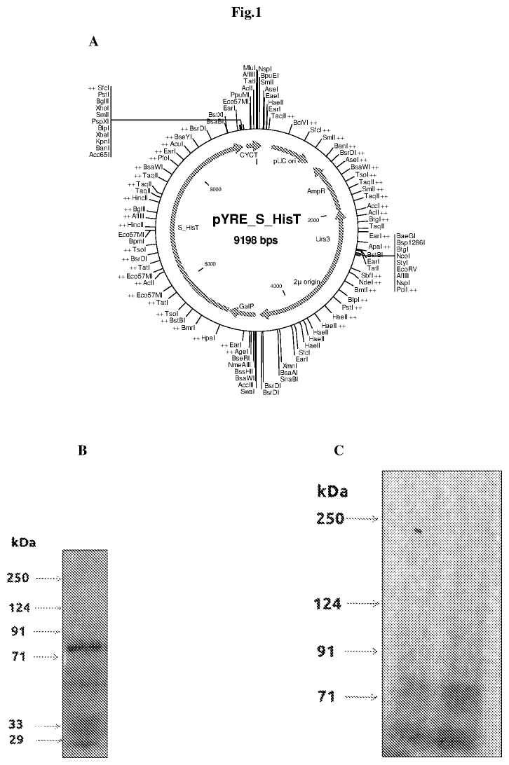

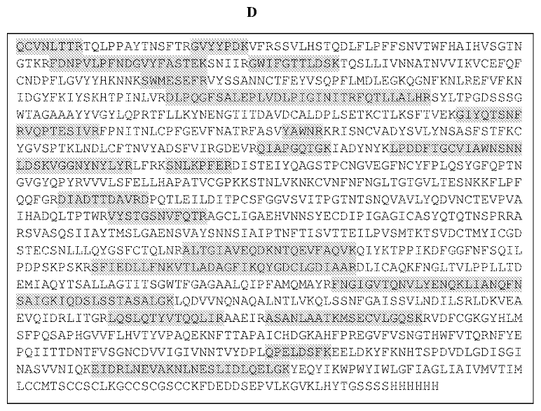

The Spike protein gene sequence was taken from reference ID strain MN908947.3 (Severe acute respiratory syndrome coronavirus 2 isolate Wuhan-Hu-1). The virus is a single stranded RNA virus with a genome size of 29903 bp. The gene was codon biased and optimized for expression in yeast host. The glycoprotein was expressed in episomal expression vector pYRE100 (FIG. 1A). The protein was His tagged for ease of analysis of expression and purification as protein.

The gene was cloned using conventional cloning methodologies and was analysed through restriction digestion. The construct was transformed into protease deficient S. cerevisiae yeast host for expression studies and further was taken for scale up and microsomes preparation for localization and analytics. Expression was characterized by anti His antibody immunoblotting, and by peptide mapping analysis. The protein can further be purified using standard purification protocol to get the purified protein.

### Example 1.1: Expression Analysis and Confirmation of Expressed Proteins Using S. cerevisiae Host

### Expression at Small Scale

The characterized recombinant construct was transformed in yeast host using Lithium acetate/SS-DNA/PEG mediated protocol known in art and transformants were selected over YNB Glucose-URA plates along with control transformed with episomal vector backbone. Few isolated healthy transformed colonies were inoculated in 10 ml YNB Glucose-URA media for episomal expression analysis. All proteins were analysed for expression in 24th hr post induced (Induction at late log phase with galactose at final concentration of 2% galactose) time point samples using anti-His antibody by Immuno-blot analysis.

### Scale Up Culture and Microsomes Preparation:

Scale up culture was performed at shake flask level at 500 ml scale was done and analysed for expression and endoplasmic localization of expressed protein by microsomal preparation. 25 ml of inoculum was prepared for respective proteins in respective media. Late log culture of the healthy grown pre-culture was inoculated into 450 ml of YPD broth in duplicates and was cultured in shake flasks for 24 hrs. The cultures were induced at a final concentration of 2% galactose for 24 hrs. Microsomal preparations were made using methodology known art. Expression and localization was analysed using His tag antibody immunoblot.

### Results

The immunoblot of His tagged S protein showed a band at app. size of 71 kDa molecular weight. This may be due to the fact that the protein is cleaved into S1 and S2 domains by Kex 2 (native protease in S. cerevisiae host) mimicking Furin cleavage site and both the fragments are migrating together at ˜71 KDa size. Further clone was taken for scale up, microsomes preparation and localization analysis. Immuno-blot results using anti His antibody from microsomal preparations also confirmed the expression of the S protein at ˜71 kDa (FIGS. 1B and C). The sample were further analysed by peptide mapping analysis to confirm identity of the protein. for correct and full-length protein expression. The peptide spanning the full-length of the protein were detected in mass spectrometry analysis (FIG. 1D). This may be due to the fact that the S1 and the S2 fragments comigrate on the gel (FIGS. 1B and C). This also further confirmed the expression of full-length S protein.

### Example 2: Expression of M Protein

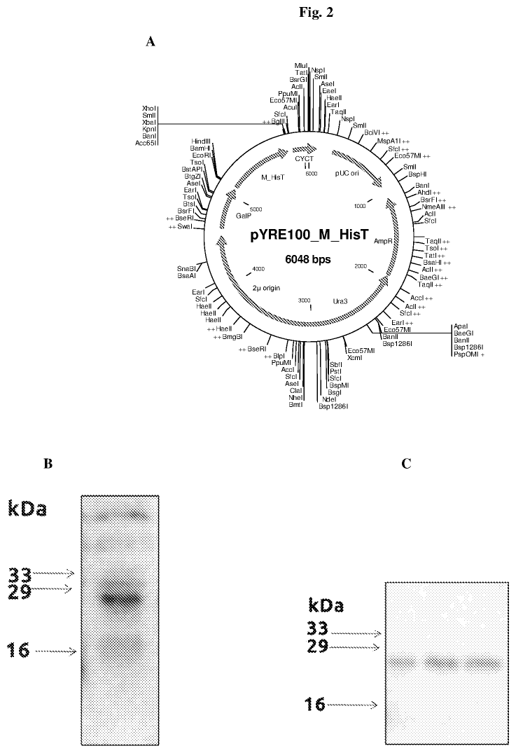

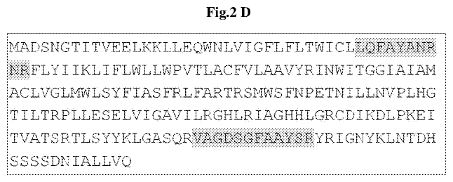

The M protein gene sequence was taken from reference ID strain MN908947.3 (Severe acute respiratory syndrome coronavirus 2 isolate Wuhan-Hu-1). The gene was codon biased and optimized for expression in yeast host. The glycoprotein was expressed in episomal expression vector pYRE100 (FIG. 2A). The protein was His tagged for ease of analysis of expression and purification as protein.

The gene was cloned, expressed and analysed as described in example 1. The final characterized construct was transformed into protease deficient S. cerevisiae yeast host for expression studies and further was taken for scale up to 500 ml and microsomes preparation for localization and analytics as mentioned for example 1. Expression was characterized by anti His antibody immunoblotting, and by peptide mapping analysis. The protein can further be purified using standard purification protocol to get the purified protein.

### Results

Immuno Blot analysis using anti His antibody showed a specific signal at expected band size of ˜26 kDa of His tagged full length M protein. Also a band at ˜31 kda was also observed which may be glycosylated form and also observed in literature. Further, Immuno-blot analysis using anti His antibody of microsomal preparation confirmed expression of full-length M protein also confirming the localization to membrane fraction (FIGS. 2B and C). The sample was further analysed using mass spectrometry to confirm protein identity. for correct and full-length protein expression. Peptide mapping results confirms the sequence identity of the His tagged M protein (FIG. 2D). In conclusion, present invention has showed and confirmed the expression of full-length surface transmembrane M structural glycoprotein of SARS CoV2 virus using S. cerevisiae host system platform.

### Example 3: Expression of Full-Length SARS CoV2 Envelope (E) Capsid Glycoprotein Antigen

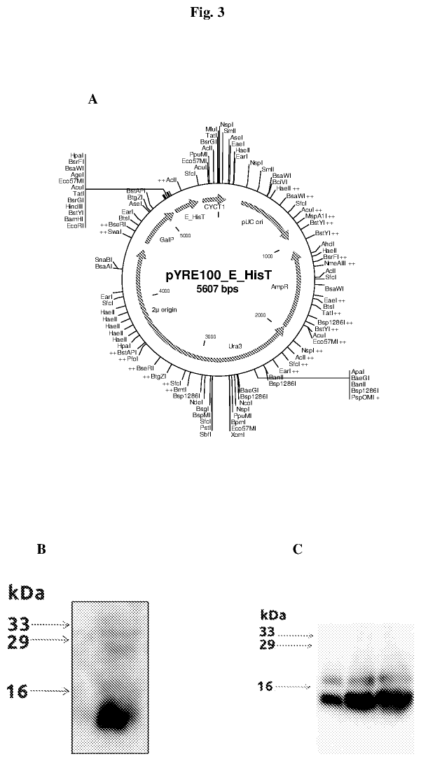

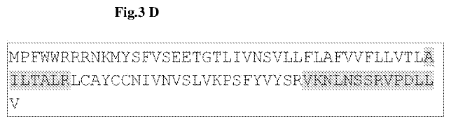

The E protein gene sequence was taken from reference ID strain MN908947.3 (Severe acute respiratory syndrome coronavirus 2 isolate Wuhan-Hu-1). The gene was codon biased and optimized for expression in yeast host. The glycoprotein was expressed in episomal expression vector pYRE100 (FIG. 3A). The protein was His tagged for ease of analysis of expression and purification as protein.

The gene was cloned, expressed and analysed as described in example 1 and 2. The final characterized construct was transformed into protease deficient S. cerevisiae yeast host for expression studies and further was taken for scale up and microsomes preparation for localization and analytics. Expression was characterized by anti His antibody immunoblotting, and by peptide mapping analysis. The protein can further be purified using standard purification protocol to get the purified protein.

### Results

Immuno Blot analysis using anti His antibody showed a signal at a size higher than the expected size of ˜9 kDa in case of full length His tagged E protein may be due to the predicted glycosylation of the envelope protein (FIG. 3B). Further the expression of protein was also observed in the microsomal preparation prepared from scale to 500 ml ((FIG. 3C). The protein was also confirmed by mass spec analysis (FIG. 3D).

### Example 4: Cloning and Expression of Multiple Antigens S, E and M of an Enveloped Virus SARS CoV-2 as Co-Expression Strategy in the Yeast Expression Host System Using Dual Approach of Episomal and Integrated Expression Together

The present examples outline the strategy for co-expression of three viral surface proteins “S”, “E” and “M”, from SARS CoV-2 virus, using yeast expression platform wherein two antigens were expressed through episomal expression and the third protein was expressed by integrating it into the yeast genome. This strategy design further provides an approach of co-expressing proteins which assemble as an enveloped VLP, maintaining structure and immunogenic potential similar to the original SARS-CoV2 virus.

### 4.1 Strategy Design

In order to co-express the proteins as VLP maintains, “S” and “E” proteins were cloned into an episomal expression vector pYRE100. “M” protein was cloned into integrative expression vector pYRI100. The gene sequences were codon biased and optimized for the cloning and expression in S. cerevisiae host with respect to certain important key critical parameters like codon usage with respect to the expression host, rare codon, GC content and AT rich or GC rich stretches optimization, internal TATA boxes, ribosomal entry sites, chi sites removal to increase the mRNA turnover and protein stability that improved the efficiency of gene expression for expression in yeast host.

### Example 4.2: Cloning and Construct Preparation

The gene for “S” and “E” proteins were cloned using conventional cloning methodologies into expression plasmid pYRE100 as two separate expression cassettes for co-expression analysis. The M protein was cloned in pYRI100 integration vector. The cloned gene were analyzed through restriction digestion. The constructs were transformed into S. cerevisiae host of recombinant expression platform for expression studies using specific antibodies by immunoblots analysis.

### Example 4.3: Transformation in S. cerevisiae Protease Deficient Host Strain

For co-expression of three proteins, the M protein gene (pYRI100_M) was integrated into protease deficient S. cerevisiae host genome and transformants were selected over selective YNB Glucose without LEU auxotrophic marker plates. This was followed by the transformation of the S and E protein genes (pYRE100_S+E episomal construct) in S. cerevisiae strain integrated with the M gene construct. Transformants were selected over selective YNB Glucose without URA and LEU auxotrophic marker plates. Vector pYRE100 plasmid was transformed in protease deficient S. cerevisiae host as control and was selected over selective YNB Glucose without URA auxotrophic marker plates. Sequential transformation was performed using Lithium acetate/SS-DNA/PEG mediated protocol by incubating the plates at 28° C. for 2-4 days for S. E, and M protein co-expression analysis.

### Example 4.4 Expression Analysis of Culture

Cultures were grown for the co expression of three proteins in YNB Glucose without URA and LEU media at 28° C. for 36 hr and control with URA media. At this stage, cultures were harvested and induced with galactose at a final concentration of 2% in YNB media without URA and LEU medium for 48 hours. Following induction, the harvested culture was used for expression analysis.

### Example 4.5 Immunoblot Analysis

In order to confirm the presence of the S. E. and M protein, immunoblot analysis was carried out using the purified VLP. For immunoblot, ˜6 μg samples were loaded on SDS-PAGE under reduced conditions. Immunoblot was developed using 1:1000 dilution of S specific antibody (Cat #ZHU1076, Sigma-Aldrich) and 1:500 dilution of poly-clonal M (Cat #GTX134866, Gene Tex) and E (Cat #MBS150849, My Bio-source) specific antibodies. Signals were detected with ECL solution (Biorad cat #170-5061).

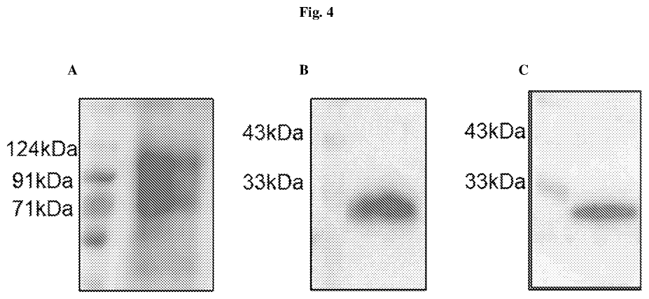

Results: Immunoblot analysis using commercially available antibodies confirmed the expression of S, E, and M proteins (FIGS. 4A, B and C). The co-expression of S. E, and M proteins was further confirmed by mass spectrometry (FIG. 5). The studies confirm that the S, cerevisiae based expression platform is able to express the three antigens which co-assemble to form a VLP (FIG. 4).

### Example 5: Simultaneous Use of the Integrated and Episomal Vectors for Co-Expression of Multiple Antigens that Assemble to Form a VLP Using Yeast Expression Platform

The three proteins (S. E and M) were expressed as three separate independent cassettes but regulated by a common promoter present in the three cassettes. The “S” and “E” proteins were cloned into an episomal expression vector pYRE100. “M” protein was cloned into integrative expression vector pYRI100. The expression of three proteins was confirmed independently as a first step, then as co-expression of S and E protein followed by co expression of all the three proteins. The constructs were transformed in protease deficient yeast host and expression was analysed. The co-expression of the three proteins was confirmed by immunoblot analysis as described previously (FIG. 4).

### Example 6: Demonstration of Immune Response Against all the Three Antigens in a Multi-Antigen Enveloped VLP for SARS CoV-2 Virus

In order to assess the immunogenicity and the antibody response against the three antigens which come together to assemble as an eVLP, specific immune responses against S. E. and M proteins were investigated by ELISA assay at week five after the initial immunization in mouse model.

To assess the antigen-specific IgG response, 96-well microtiter plates were coated with 0.25 μg of purified S. E and M proteins were individually at 2-8° C. overnight and blocked with 2% BSA for 1 h at room temperature. Diluted sera (1:1000) were applied to each well and incubated for at 37° C. for 2 h, followed by incubation with HRP conjugated goat anti-mouse antibodies at 37° C. for 1 h. The plate was developed using TMB, following 2 M H₂SO₄ addition to stop the reaction, and read at 450 nm using an ELISA plate reader.

### Results

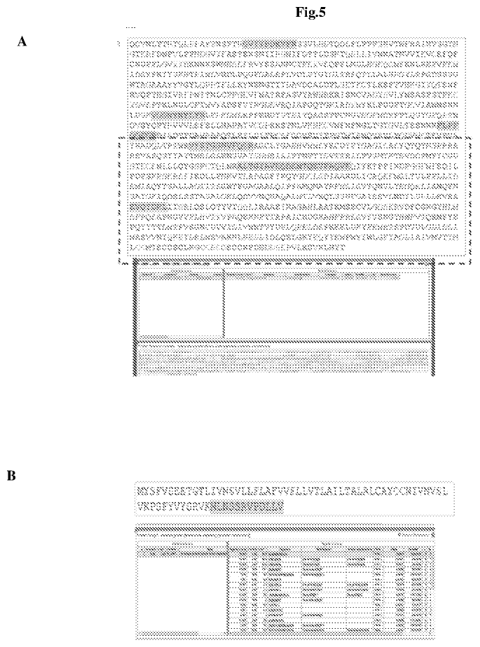

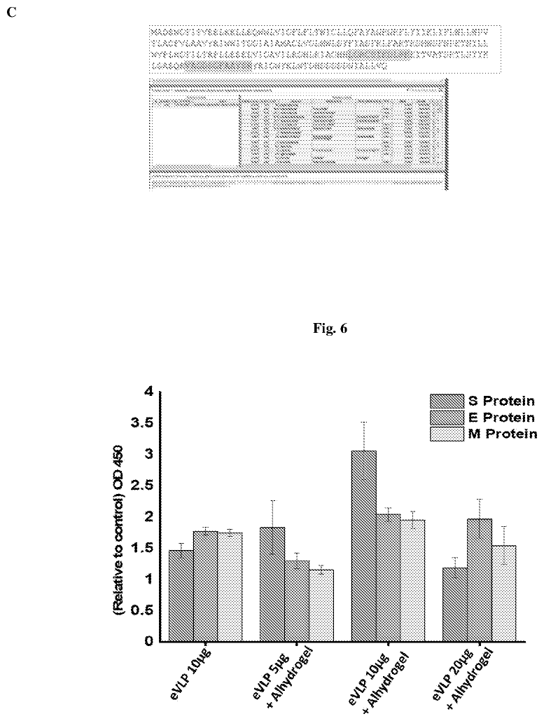

Evaluation of immune response against the S. E and M proteins showed that mice vaccinated with or without an adjuvant (Alhydrogel was used as an adjuvant in the studies) could surmount a immune response against the three eVLP antigens (FIG. 6). Thus, these results highlight the potential of eVLP in inducing a strong and potent immune response by eliciting effective antibody responses for all three antigens and, therefore, may serve as an effective vaccine candidate for novel coronavirus infections. This eVLP based vaccine could be helpful to also prevent infections from the various variants of the SARS-CoV-2 which may have developed mutations in the S protein.

### Example 7: The Multi-Antigen Enveloped VLP Produced in Yeast Expression System is Highly Immunogenic as Demonstrated by In Vivo Studies

This example shows that a triple antigen, eVLP is able to surmount an immune response with high titers in mice. Also, the antibodies are seen to be neutralizing in nature.

### Example 7.1: Humoral Immune Responses and Immunization in Mice

BALB/c mice were immunized with eVLP formulation with and without adjuvant Alhydrogel (AH). Adjuvant groups included injection with 5 μg eVLP+AH, 10 μg eVLP+AH and 20 μg eVLP+AH. The eVLP alone group was injected with 10 μg eVLP. Two controls groups were set with buffer alone injection and the with adjuvant AH only injection. The animals were injected at 14 days interval (Day 0, 14, 28) with one primary and two booster doses.

### Example 7.2: Serum Antibody Measurements

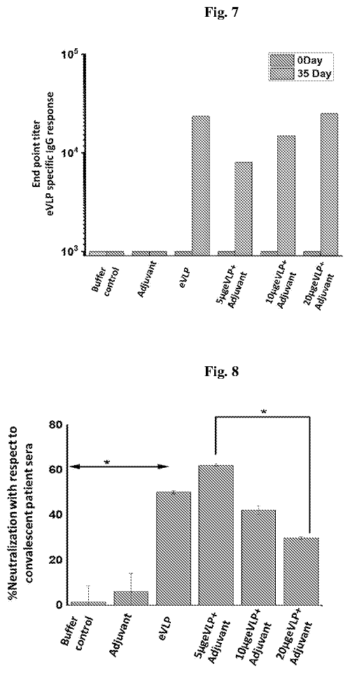

IgG mediated antibody response and titer of serum samples collected from immunized animal sera at 0 and 35^{th} day were determined by enzyme-linked immunosorbent assay (ELISA). Briefly, 96 well ELISA plate was coated with 0.25 μg of eVLP at 2-8° C. overnight and blocked with 2% BSA for 1 h at room temperature. Serum dilutions were applied to the well and incubated for at 37° C. for 2 h, followed by incubation with HRP conjugated goat anti-mouse antibodies at 37° C. for 1 h. The plate was developed using TMB, following 2 M H₂SO₄ addition to stop the reaction, and read at 450 nm using an ELISA plate reader (FIG. 7).

### Example 7.3: SARS-CoV-2 Neutralization Assays

Neutralization experiments on serum samples of immunized animals and convalescent patients were performed as per the manufacturer protocol (sVNT Kit, GenScript, USA). Briefly, the samples were pre-incubated with HRP-RBD to bind the circulating neutralizing antibodies to HRP-RBD. The mixture was added to an hACE2 protein pre-coated plate and incubated at 37° C. for 15 min. The wells were washed 4 times with PBS and the mixture was incubated with TMB at 20-25° C. in the dark for 15 min. The stop solution was added, and the plate was read at 450 nm in a microtiter plate reader. Neutralisation potential of mice was plotted relative to human neutralization potential (reference), taken as 100 percent (FIG. 8).

### Example 7.4: Plaque Reduction Neutralization Test (PRNT)

For the PRNT assay, Vero E6 cells (1×10⁵ per well) were seeded onto 24-well plates. On the following day, 30 PFU of infectious wild-type SARS-CoV-2 was incubated with diluted serum (total volume of 150 μl) at 37° C. for 1 h. The virus-serum mixture was added to the pre-seeded Vero E6 cells and incubated at 37° C. for 1 h. Following this, 1 ml of 2% Modified Eagle's Medium (MEM) containing carboxy methylcellulose (CMC) was added to the infected cells. After 3 days of incubation, 1 ml 3.7% formaldehyde was added and incubated at room temperature for 30 min. The formaldehyde solution from each well was discarded, and the cell monolayer was stained with a crystal violet solution for 60 min. After washing with water, plaques were counted for PRNT50 calculation. The PRNT assay was performed in a BSL-3 facility.

### Results

The total IgG response was evaluated in different mice groups using the sera samples at day 0 and 35. The total IgG binding endpoint titers (EPTs) from all the immunized mice groups were measured against eVLP. Results showed that all the eVLP vaccinated groups elicited IgG-mediated response as compared to controls (FIG. 7). A dose-dependent increase in titer was observed. Interestingly, the eVLP was seen to be immunogenic even without the adjuvant suggesting its potential to generate an immune response.

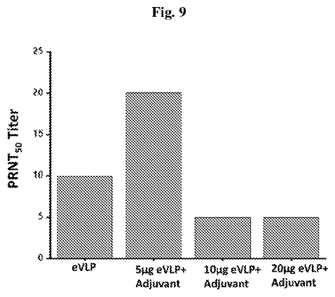

RBD-specific neutralization inhibition was evaluated in eVLP immunized animals and compared to that with convalescent patients' sera. Neutralization potential (NP) of convalescent patients' sera was used as a reference and considered 100 percent; data from mice was plotted relative to that. In the neutralization inhibition assay, sera from all the eVLP vaccinated groups showed significant NP by inhibiting the RBD and ACE2 interaction compared to control groups (FIG. 8). However, maximum inhibition of RBD and ACE2 cell surface receptors was observed at the lowest (5 μg) dose of adjuvanted eVLP (62.3±0.50%) as compared to control groups (AH: 6.12±8.06%; FIG. 9).

PRNT is considered as the gold-standard for determining immune protection. To validate the virus neutralization assay results, a conventional PRNT assay was performed for the vaccinated groups. On day 3 post-challenge, wild-type virus neutralizing activity capable of reducing SARS-CoV-2 infectivity by 50% or more (PRNT50) was detected in all eVLP vaccinated groups (FIG. 6). The serum dilutions yielding 50% virus neutralization (PRNT50 titers) were higher in eVLP (1:10) and AH plus 5 μg eVLP (1:20) groups in comparison to other adjuvanted groups, where PRNT50 was observed at a dilution of 1:5 (FIG. 9).

The results thus comprehensively show that eVLP was able to surmount an immune response which has neutralization potential.

### Example 8: The eVLP Produced Using Yeast Based Expression Platform Mimics the Viral Epitopes and Produces a Cellular Immunity Response

To assess if the eVLP mimics the viral epitopes, it was used in cytokine assays from healthy and convalescent human PBMCs. VLP specific lymphocyte proliferation was evaluated by ELISA assay using sera from convalescent patients (N=5) and healthy controls sera (N=2). All the human samples have been taken after adequate ethical committee approval.

### Example 8.1: Determination of Cell Proliferation Using BrDU Assay

PBMCs were separated from the blood of the convalescent patients and stimulated with two doses of the VLP (2.5 μg and 5 μg). The proliferation rate of lymphocytes was measured using 5-Bromo-2-deoxyuridine (BrDU) assay as per the manufacturer instructions (Cell Proliferation ELISA BrDU, Roche, USA).

### Example 8.2: Cytokine Profiling of Stimulated PBMCs

Healthy control and patient PBMCs (1×105 cells/well) were stimulated with two doses of the VLP formulation (2.5 μg and 5 μg) at 37° C. in a humified chamber containing 8% CO2 for 120 h. Supernatants were collected, and cytokine staining for IFN-g was performed as per the manufacturer instructions (Human IFN-g ELISA set (RUO), BD Biosciences, USA).

### Results

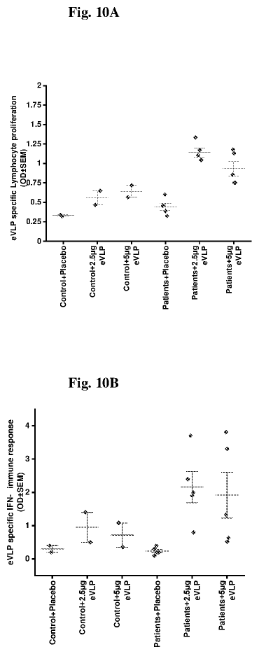

BrDU cell proliferation studies of PBMCs showed a higher lymphocyte proliferation in patient samples subjected to two doses of VLP formulation (2.5 μg-2 Fold; 5 μg:1.45 Fold) in comparison to PBMCs from healthy individuals (FIG. 10A). Further, elevated levels of IFN-γ in the VLP stimulated patients PBMCs (2.16±0.33) was observed as compared to healthy controls (0.95±0.23), suggesting induction of T helper 1 (Th1)-biased cellular immune responses (FIG. 10B).

The results demonstrate that the VLP formulation containing S. E and M full length proteins from SARS CoV-2 virus, can mimic the epitopes of the original SARS-CoV2 virus, as it can stimulate a cell mediated immune response with PBMCs from convalescent patients which have never been exposed to VLP formulation. In literature, none of the vaccine candidates have shown a recall of cellular immune response from convalescent patients confirming the mimicking epitope by a potential vaccine candidate.

### Example 9: Safety of the Vaccine Formulation Over Large Range of Dose Formulation

Extensive studies were performed to demonstrate that the enveloped VLP for SARS CoV-2 virus, composed of three antigens, S. E and M, and produced using the yeast expression platform is safe over a large range of doses. Two rodent species have been used to demonstrate the safety of this formulation: rats and mice.

### Example 9.1: Studies in Mice (Mus musculus)

Multiple cohorts of Mice (Mus musculus sp.), Strain Balb/c, gender male, age 6-8 weeks, were taken and under all applicable guidelines, injected intramuscularly with liquid formulation of the VLP in dose of 5 μg, 10 μg, 20 μg, 50 μg, 100 μg and 150 μg. Two vaccine formulations were studied simultaneously: a) with commercially available adjuvant aluminum hydroxide gel and b) VLP alone with no adjuvant. In each cohort, five animals were included to maintain statistical significance of the data observed.

The mice were given three doses, on day 0, day 14 and day 28 and observed for any signs/symptoms of morbidity or other changes. No mortality was documented through the course of study. All animals looked healthy post injection, and all through the course of study, there was no visible changes in their motility, no behavioral changes inside the cage were observed or fighting wounds or any skin infections were observed, no lack of shininess of hair was observed. Food habits were observed to be normal for all the groups.

### Example 9.2: Studies in Mice (Swiss Albino): Abnormal Toxicity Studies

Abnormal toxicity test with the VLP as vaccine candidate in Swiss albino mice was tested in 5 male mice. The abnormal toxicity test was performed as per Indian Pharmacopoeia IP 2018 and OECD principles on Good Laboratory Practice.

The test was performed with 5 animals. 250 μL of the test candidate, VLP in liquid formulation was reconstituted to the 250 μL of the adjuvant (Alhydrogel) and mixed/shook well for one minute. To each mouse, 500 μL of the reconstituted test item was administered immediately via intraperitoneal route.

The animals were observed for 7 days for any adverse effects or clinical signs and recorded at 30 min, 1, 2, 4, 24 hr and then after once a day till the termination/completion of the experiment. All animals were normal, and no signs of clinical toxicity were observed throughout the experiment. All the animals used in this study gained body weight on day 7 when compared to its respective day 0 (dosing start day).

The animals were humanely sacrificed by carbon dioxide asphyxiation at termination of the experiment.

Based on the results obtained, it was concluded that the formulated eVLP, with the given Adjuvant, passed the abnormal toxicity test and was classified as “Non-toxic” to the mice under the conditions of the study conducted (Table 1).

TABLE 1
Abnormal Toxicity studies in Swiss albino

| | Days of Observation | | | | | | | | | | | | | | | |
|---|---|---|---|---|---|---|---|---|---|---|---|---|---|---|---|---|
| | Acclimatization Phase | | | | | | | | Treatment Phase | | | | | | | |
| A.No | 1 | 2 | 3 | 4 | 5 | 6 | 7 | 8 | 0 | 1 | 2 | 3 | 4 | 5 | 6 | 7 |
| 01 | 1 | 1 | 1 | 1 | 1 | 1 | 1 | 1 | 1 | 1 | 1 | 1 | 1 | 1 | 1 | 1 |
| 02 | 1 | 1 | 1 | 1 | 1 | 1 | 1 | 1 | 1 | 1 | 1 | 1 | 1 | 1 | 1 | 1 |
| 03 | 1 | 1 | 1 | 1 | 1 | 1 | 1 | 1 | 1 | 1 | 1 | 1 | 1 | 1 | 1 | 1 |
| 04 | 1 | 1 | 1 | 1 | 1 | 1 | 1 | 1 | 1 | 1 | 1 | 1 | 1 | 1 | 1 | 1 |
| 05 | 1 | 1 | 1 | 1 | 1 | 1 | 1 | 1 | 1 | 1 | 1 | 1 | 1 | 1 | 1 | 1 |

18.1 Individual body weight and % body weight change

| | | Acclima- | Experi- | Experi- | |
|---|---|---|---|---|---|
| | | tization | ment | ment | % Body |
| | | start | start | completion | weight |
| | Group/ | day | day | day | change |
| A.No. | Sex | (g) | (g) | (g) | ( Day 0-7) |
| 01 | Initial test/ | 18.08 | 19.18 | 19.97 | 4.12 |
| 02 | Male | 17.20 | 18.03 | 18.75 | 3.99 |
| 03 | | 18.24 | 18.97 | 19.76 | 4.16 |
| 04 | | 18.50 | 19.27 | 20.04 | 4.00 |
| 05 | | 18.39 | 19.23 | 19.98 | 3.90 |

Note:
1—Normal.
Note:
A.No.—Animal number; g—Grams; %—Percent.

### Example 9.3: Study in Guinea Pigs: Abnormal Toxicity Studies

Abnormal toxicity test with the VLP formulation as a vaccine candidate was conducted in another species, using the Guinea Pigs model. The formulation was tested in 2 male guinea Pigs. The abnormal toxicity test was performed as per Indian Pharmacopoeia IP 2018 and OECD principles on Good Laboratory Practice (revised 1997, issued January 1998).

The test was performed with 2 animals. 250 μL of the test candidate, VLP in liquid formulation was reconstituted to the 250 μL of the adjuvant (Alhydrogel) and mixed/shook well for one minute. To each mouse, 500 μL of the reconstituted test item was administered immediately via intraperitoneal route.

The animals were observed for 7 days for any adverse effects or clinical signs and recorded at 30 min, 1, 2, 4, 24 hr and then after once a day till the termination/completion of the experiment. All animals were normal, and no signs of clinical toxicity were observed throughout the experiment. All the animals used in this study gained body weight on day 7 when compared to its respective day 0 (dosing start day). The animals were humanely sacrificed by carbon dioxide asphyxiation at termination of the experiment.

### Results

Based on the results obtained, it was observed that the formulated test item as a vaccine candidate with the given Adjuvant passed the abnormal toxicity test and was classified as “Non-toxic” to the guinea pigs under the conditions of study conducted (Table 2).

TABLE 2
Abnormal Toxicity studies in Guinea pigs
Clinical signs

| | Days of Observation | | | | | | | | | | | | | | | |
|---|---|---|---|---|---|---|---|---|---|---|---|---|---|---|---|---|
| | Acclimatization Phase | | | | | | | | Treatment Phase | | | | | | | |
| A. No | 1 | 2 | 3 | 4 | 5 | 6 | 7 | 8 | 0 | 1 | 2 | 3 | 4 | 5 | 6 | 7 |
| 01 | 1 | 1 | 1 | 1 | 1 | 1 | 1 | 1 | 1 | 1 | 1 | 1 | 1 | 1 | 1 | 1 |
| 02 | 1 | 1 | 1 | 1 | 1 | 1 | 1 | 1 | 1 | 1 | 1 | 1 | 1 | 1 | 1 | 1 |

Note:
1—Normal.

Individual body weight and % body weight change

| | | Acclimatization | Experiment | Experiment | % Body |
|---|---|---|---|---|---|
| | | start | start | completion | weight |
| | Group/ | day | day | day | change |
| A. No. | Sex | (g) | (g) | (g) | (Day 0-7) |
| 01 | Initial test/ | 307.02 | 324.77 | 341.08 | 5.02 |
| 02 | Male | 301.60 | 318.54 | 339.67 | 6.63 |

Note:
A. No.—Animal number; g—Grams; %—Percent.

### Example 10: Stoichiometric Ration Calculation for S, M and E in the Produced VLP from Co Expression

Relative amounts of S, M and E proteins were calculated using quantitative ELISA methodology. To estimate the individual amount of protein present in VLP, ELISA plate wells were coated with known amounts of respective purified standard S1, E and M proteins to generate a standard curve for each protein.

Similarly, purified VLP was coated for the analysis of number of molecules of S, M and E protein in assembled VLP. Relative quantitative estimation of S:M:E ratio in the VLP was measured against purified protein standard curve generation.

### 10.1: ELISA

Purified standard proteins (S1, M and E) and VLP were coated at different amount per well on ELISA plate for 14 hr at 4° C. 1% BSA was used as blocking solution. The proteins were then detected in ELISA using respective commercially available antibodies along with the VLP.

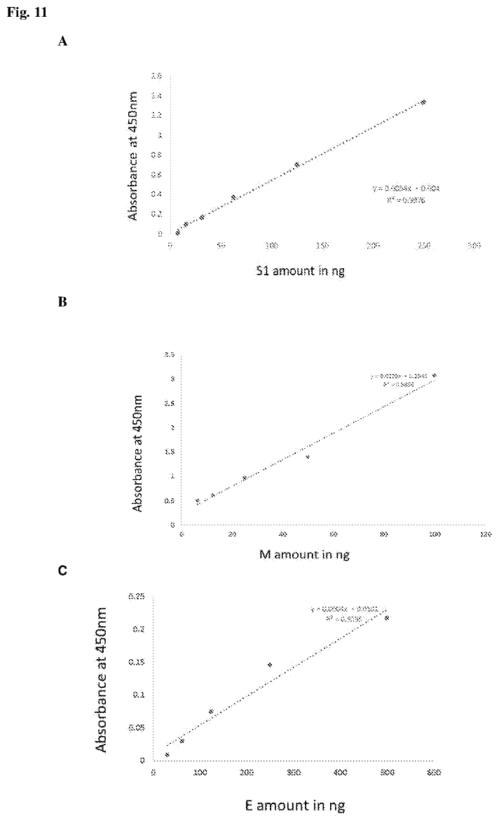

For S1 standard protein, RBD specific antibody (dilution 1:250); for M standard protein, M specific antibody (1:500); for E standard protein, E specific antibody (1:250) were used along with VLP coated wells. After washing, respective secondary antibodies were added. Finally, absorbance measured with TMB solution at 450 nm. Graphs were plotted accordingly and mentioned in FIGS. 11A, B and C.

### Results

### Calculations for Estimation of S1, M and E

A linear plot of OD vs the amount of the protein coated was plotted from which the equation of straight line was generated (y=m₁x+c). Using the derived equation, the amount of protein (x) in the VLP was calculated using the absorbance value. Further, using the protein amounts the number of molecules of each protein was determined using online software (https://www.bioline.com/media/calculator/01_04.html), (Table 3).

TABLE 3
Calculations for S and M molecules in assembled VLP sample:

| Antibody used | | Amount of | Mol wt | Number of |
|---|---|---|---|---|
| for detection | Absorbance | protein (ng) | (kDa) | molecules |
| RBD | 0.063 | 10.9 | 420 | 1.56 × 10¹⁰ |
| M specific | 0.426 | 6.29 | 25 | 1.51 × 10¹¹ |
| E specific | 0.0104 | 0.812 | 54 | 0.8 × 10¹⁰ |

Obtained values showed S:M:E stoichiometric value of 1:9.7:0.5 in VLP. The ratios were calculated for VLPs which showed the expected range of SARS-CoV-2 virion size of 180 to 200 nm However, due to pleomorphic nature of the Corona viruses the ratio and size of the VLP is expected to vary. The M glycoprotein of coronaviruses drives the assembly hence is the most abundant viral structural protein in the virion and oligomerizes and interacts laterally with S and E, the other two viral membrane proteins to make VLP while E protein which in literature is described to be present in low amounts but important for assembly.

### Example 11: Production of the Scalable Amount of VLPs

The VLPs were produced in a transformed yeast cell culture and cultivated in a batch or fed-batch cultivation system using a cultivation medium which is supplemented with at least one or more additives to support high density culture. Transformed yeast cell was further grown in a culture to a stage of log phase growth with a media supplemented with additives, such as glucose, glycerol, either at the time of inoculation or later at the time when the cells are grown to a high cell density. The transformed yeast cells are grown to a density of up to 60 at OD₆₀₀ and up to 120-300 g/L, WCW in a time interval of 12-24 hours at a growth temperature of 28-32° C. after which the temperature is cooled to 25-28° C. followed by supplementing the media with an amount of an induction agent such as galactose, glycerol or a mixture of both and boosting solution containing peptides, amino acids, tryptone and yeast extract in an amount of 10× concentrated 12 g/L tryptone and 24 g/L yeast extract. The cultivation media is saturated by air to a level 20-80% saturation and incubated for period induction of high protein expression, by an additive at 48-120 hours at 25-28° C. The cells were harvested either by centrifugation step, or a microfiltration step, or a mixture of both resulting into VLPs and cultivation medium.

The VLPs were washed and buffer was changed to a lysis buffer for cell lysis, by a diafiltration or buffer exchange or an ultrafiltration step, or a mixture of both to resuspend the yeast cells in the lysis buffer. The lysis buffer contains 20 mM-100 mM Potassium phosphate pH 7.2, 0.0005%-0.001% Tween 80 or Tween 20 or a non-ionic detergent, 2 mM PMSF and is also supplemented with a nuclease where the nuclease treatment was given for 2-18 hours at 2-8° C., or 37° C., preferably, for 2-3 hours at 37° C. or 4-18 at 2-8° C.

The yeast cells containing the VLP in lysis buffer was disrupted by mechanical force or ultrasonic waves, or a French press or a method of cell lysis, or a combination of the above and results into the lysed or broken yeast cells where lysis outcome is up to 60-100% lysed cells in the lysis buffer containing the nuclease. The yeast cells containing the VLP in lysis buffer was further disrupted by mechanical force, specifically with a high-pressure mechanical homogenizer, where the yeast cells were subjected to a mechanical pressure of 200-800 Bar, at 2-8° C., and the process is repeated 2-8 times or passes to obtain a lysis outcome of 60-100% lysed cells in the lysis buffer. The method results into the VLPs, from the nuclease treated lysis buffer and produce partially isolated VLP separating the lysis buffers, nucleases and other contaminating components.

The exchange buffer solution consists of 20-100 mM Potassium phosphate pH 7.2, 0.0005%-0.001% Tween 80) and nuclease treated VLP in lysis buffer is buffer exchanged and diafiltered using 400-750 kDa hollow fiber cartridge of I.D of 0.5-1.0 mm.

The VLPs were purified using an anion or cation ion exchange resin or a mix mode resin or a combination of the two. A mix mode resin, Capto Core 700 was used in the present invention. The VLP material was loaded and eluted with a buffer containing up to 1.5M KCl and collection of fractions was performed based on A280 and VLP purity. The VLPs were further taken to dialysis and buffer exchange step with a formulation buffer, consisting of 20-100 mM Potassium phosphate pH 7.2 supplemented with 50-100 mM KCl, 0.0005-0.001% Tween 80 and 2-10% Sucrose. Dialyzed material was sterile filtered using 0.2 μm filter and tested for yield and the yield was determined to be 60 mg-120 mg/L of purified VLP.

The present invention provides VLPs and method of preparing said VLPs for SARS-CoV which are stable and display appropriate stoichiometric and are capable of inducing immunogenic response. The present invention also provides the strategy for efficient expression of the SARS-CoV proteins (S, M and E) individually and in combination. The VLPs are suitable for incorporated in an efficient vaccine against SARS-CoV.

Advantages of the present invention:
Recombinant VLPs have significant morphological resemblance to the parent virus, provide a highly repetitive immunogenic surface structure, and the retention of cell uptake and immune processing pathways associated with their parent virus. They themselves are devoid of the parent virus genome and hence are non-pathogenic or incapable of infection.
The SARS CoV-2 VLP described in this invention can be bulk expressed in bioreactor cultures, and using the current expression system and processes, it is capable of millions of doses in a very short span of time, utilizing advanced in vitro protein expression systems for producing vaccine-grade VLP suitable for clinical administration.
Multi-antigen VLP vaccine of the present invention is capable of providing antibody response against diverse antigens and has ability to recognise the virus even in case of mutations, which is a very important aspect that is not being addressed by vaccines available or under development currently.
The enveloped VLPs have been difficult to express and purify from yeast-based platform in large quantities; this invention describes the process and method to do so, which can also be helpful in developing vaccines for other similar viruses and infections, both in human and animal.
The potential vaccine candidate in this invention has demonstrated in animal models, safety, simplicity of production and favourable immunological characteristics to induce both humoral and cellular immune responses. The SARS cov-2 virus poses a continuous threat to human health in the pandemic and with multiple variations, which can be addressed by the current invention.
Affordability of the dose is an important criterion for any vaccine to be successfully developed and launched; with the current processes and high yields described in this application, the vaccine cost per dose can be made highly affordable.

What is claimed is:

1. A recombinant yeast cell,
wherein the yeast cell expresses a polynucleotide with which it has been transformed that encodes a polypeptide having an amino acid sequence of a protein that is a Spike (S), Membrane (M), or Envelope (E) protein of a member of the SARS-CoV-2 family, or that encodes a variant of the S, M or E protein;
and
wherein the S, M or E protein, or the variant thereof, are either individually expressed or co-expressed.

2. The recombinant yeast cell as claimed in claim 1, wherein the amino acid sequence of the S, M or E polypeptide is that of SEQ ID NO 1, SEQ ID NO 2, or SEQ ID NO 3, respectively, or is a variant of that sequence that has at least 70% amino acid sequence identity to SEQ ID NOs 1, 2, or 3.

3. The recombinant polypeptides yeast cell as claimed in claim 1, wherein the polynucleotide has been codon optimized.

4. The recombinant yeast cell as claimed in claim 1, wherein the yeast cell expresses an S protein having the amino acid sequence of SEQ ID NO 1 or a variant thereof.

5. The recombinant yeast cell as claimed in claim 4, wherein the S protein that is expressed is a full-length S protein or variant thereof that is a single pass type I membrane glycoprotein.

6. The recombinant yeast cell as claimed in claim 1, wherein the yeast cell expresses a M protein having the amino acid sequence of SEQ ID NO 2 or a variant thereof.

7. The recombinant yeast cell as claimed in claim 6, wherein the M protein that is expressed is a full-length M protein or variant thereof that is a multi-pass membrane glycoprotein.

8. The recombinant yeast cell as claimed in claim 1, wherein the yeast cell expresses an E protein having the amino acid sequence of SEQ ID NO 3 or a variant thereof.

9. The recombinant yeast cell as claimed in claim 8, wherein the E protein that is expressed is a full-length E protein or variant thereof that is a single pass type III membrane glycoprotein.

10. The recombinant yeast cell as claimed in claim 1, wherein the yeast cell co-expresses the S, M, and E proteins, or variants thereof, and wherein the co-expressed S, M and E proteins or variants thereof assemble into complexes in the yeast cell in which the S, M, and E proteins, or variants thereof, form ordered aggregates.

11. The recombinant yeast cell as claimed in claim 10, wherein the ordered aggregates comprise Virus like particles (VLPs).

12. The recombinant yeast cell as claimed in claim 1, wherein the yeast cell has been transformed with one or more polynucleotides having the sequence of SEQ ID Nos 4, 5, or 6 or a variant thereof.

13. The recombinant yeast cell as claimed in claim 12, wherein the polynucleotide sequence of the variant has at least 60% sequence identity to the polynucleotide sequence of SEQ ID NOs 4, 5, or 6.

14. The recombinant yeast cell as claimed in claim 12, wherein the polynucleotide sequence has been codon biased and optimized.

15. The recombinant yeast cell as claimed in claim 12, wherein the cell has been transformed with a gene having a sequence comprising SEQ ID NO 4 or a variant thereof encoding an S protein.

16. The recombinant yeast cell as claimed in claim 12, wherein the cell has been transformed with a gene having a sequence comprising SEQ ID NO 5 or a variant thereof encoding an M protein.

17. The recombinant yeast cell as claimed in claim 12, wherein the cell has been transformed with a gene having a sequence comprising SEQ ID NO 6 or a variant thereof encoding an E protein.

18. The recombinant yeast cell as claimed in claim 1, wherein the sequence of the S, M or E protein is that of the SARS-CoV-2 S, M or E protein.

19. A method of expressing SARS-CoV proteins comprising the steps of:
i. selecting target genes from the group consisting of genes that encode SARS-CoV S, E, and M proteins;
ii. optimizing and codon biasing the target gene sequences;
iii. preparing a construct that comprises a target gene or genes of part ii inserted into a yeast-based expression vector by cloning the target genes individually or in combination into the expression vector, wherein the expression vector is a yeast-based expression vector that is an episomal expression vector or an integrative expression vector;
iv. transforming the construct of part iii into a protease deficient yeast host cell; and
v. expressing the SARS-CoV proteins.

20. The method as claimed in claim 19, wherein the target gene encodes the S protein from SARS-CoV.

21. The method as claimed in claim 19, wherein the target gene encodes the M protein from SARS-CoV.

22. The method as claimed in claim 19, wherein the target gene encodes the E protein from SARS-CoV.

23. The method of expressing SARS-CoV proteins as claimed in claim 19, wherein the method comprises the steps of:
i. selecting genes encoding S, E and M proteins from SARS-CoV as target genes;
ii. optimizing and codon biasing the target gene sequences encoding the selected S, E and M proteins;
iii. cloning polynucleotides encoding the S protein and the E protein into a yeast episomal expression vector to obtain a construct having SEQ ID NO 10 and cloning a polynucleotide encoding the M protein into a yeast integrative expression vector to obtain a construct having SEQ ID NO 11;
iv. transforming both constructs into the same yeast host cell;
v. co-expressing the SARS-CoV S, E and M proteins in the yeast host cell.

24. The method as claimed in claim 19, wherein all three proteins S, M and E, or variants thereof, are either co-expressed individually from an episomal vector or from an integrative vector, or are co-expressed from a combinations thereof.

25. The method as claimed in claim 19, wherein the proteins that are expressed comprise the S, M or E Corona virus proteins.

26. The method of expressing SARS-CoV proteins as claimed in claim 19, wherein the yeast host is Saccharomyces cerevisiae.

27. A recombinant yeast host that has been transformed with a target gene or genes selected from genes that encode the S, M and E proteins of SARS-CoV and comprising the sequence of SEQ ID Nos 7, 8 and 9, respectively, or variants thereof, where in the target genes are inserted into yeast expression vectors, wherein the vectors are selected from episomal and/or integrative vectors and wherein any two of the S, M or E proteins are expressed from an episomal vector and one of the S, M and E proteins is expressed from an integrative expression vector.

28. The recombinant yeast host as claimed in claim 27, comprising a polynucleotide having the sequence of SEQ ID NO 7 that encodes the S protein in a yeast episomal expression vector.

29. The recombinant yeast host as claimed in claim 27, comprising a polynucleotide having the sequence of SEQ ID NO 8, that encodes the M protein in a yeast integrative or episomal expression vector.

30. The recombinant yeast host as claimed in claim 27, comprising a polynucleotide having the sequence of SEQ ID NO 9, that encodes the E protein in a yeast episomal or integrative expression vector.

31. The recombinant yeast host as claimed in claim 27, wherein the construct is a multiprotein construct comprising polynucleotides encoding S and E proteins expressed from an episomal expression vector to obtain a construct having SEQ ID NO 10 and a construct encoding the “M” protein expressed from an integrative expression vector to obtain a construct having SEQ ID NO 10.

32. A composition that comprises recombinant corona virus like particles (VLPs) in which the stoichiometric value of the S:M:E proteins in the particles is 1:9.7:0.5, and wherein:
(i) S is a SARS-CoV-virus Spike protein or variant thereof;
(ii) M is a SARS-CoV-virus Membrane protein or variant thereof; and
(iii) E is a SARS-CoV-virus Envelope protein or variant thereof.

33. The composition as claimed in claim 32, wherein the VLPs are in a yeast cell and the VLPs comprise one or more S, M and E proteins encoded by SEQ IDs selected from SEQ ID NO 1, 2 and 3, respectively or variants thereof.

34. The composition as claimed in claim 32, wherein the VLPs maintain a size range and stoichiometry range for all the co-expressed proteins.

35. A method of preparing target VLPs comprising target proteins selected from S, M and E proteins of SARS-CoV, individually or in combination, the method comprising the steps of:
i. inserting the target gene selected from S, M and E proteins of SARS-CoV into protease deficient yeast host cell by either episomal construct or integrative construct;
ii. transforming the selected genes in protease deficient yeast host strain;
iii. selecting the transformants on selective Yeast Nitrogen Base (YNB) Glucose medium and without LEU auxotrophic marker in case of integrative construct and without URA auxotrophic marker in case of episomal construct;
iv. transforming the selected genes in protease deficient yeast host strain;
v. selecting the transformants on YNB with Glucose without URA and LEU auxotrophic marker plates;
vi. performing transformation;
wherein, transformation is a sequential transformation and performed using Lithium acetate/SS-DNA/PEG mediated protocol.

36. The method of preparing target VLPs as claimed in claim 35, comprising the target M protein of SARS-CoV, individually, comprising the steps of:
i. Integrating/episomally inserting the M gene into protease deficient yeast host cell;
ii. transforming the M gene in Protease deficient yeast host strain;
iii. selecting the transformants on Yeast Nitrogen Base (YNB) Glucose medium without LEU auxotrophic marker in case of integrative construct and without URA auxotrophic marker in case of episomal construct;
iv. performing transformation;
wherein, transformation is performed using Lithium acetate/SS-DNA/PEG mediated protocol.

37. The method of preparing the target VLPs as claimed in claim 35, comprising the target E and M protein of SARS-CoV, in combination, comprising the steps of:
i. integrating/episomally inserting the E or M gene into protease deficient yeast host cell;
ii. transforming the E or M gene in Protease deficient yeast host strain;
iii. selecting the transformants on Yeast Nitrogen Base (YNB) Glucose medium without LEU auxotrophic marker in case of integrative construct and without URA auxotrophic marker in case of episomal construct;
iv. transforming the E or M gene in Protease deficient yeast host strain for co-expression;
v. selecting the transformants on YNB Glucose without URA and LEU auxotrophic marker plates for co-expression using episomal and integration vector; and selecting the transformants on YNB Glucose without URA for expression of both using episomal vector or YNB Glucose without LEU for expression of both using integration vector; and
vi. performing transformation;
wherein transformation is performed using Lithium acetate/SS-DNA/PEG mediated protocol.

38. The method of preparing the target VLPs as claimed in claim 35, comprising the target proteins selected from S, M and E proteins of SARS-CoV, in combination, comprising the steps of:
i. inserting the target gene selected from S, M and E proteins of SARS-CoV into protease deficient yeast host cells;
ii. selecting the transformants on selective Yeast Nitrogen Base (YNB) Glucose medium and without LEU auxotrophic marker;
iii. transforming the selected genes in protease deficient yeast host strain;
iv. selecting the transformants on YNB with Glucose without URA and LEU auxotrophic marker plates;
v. performing transformation;
wherein the transformation is a sequential transformation and is performed using Lithium acetate/SS-DNA/PEG mediated protocol by incubating the plates at 28° C. for 2-4 days for S, E, and M proteins and cultures were grown in YNB Glucose without URA and LEU media at 28° C. for 36 hr and control with URA media.

39. A method of producing the scalable amount of VLPs comprising the steps of:
i. overexpressing recombinant proteins or VLPs in a transformed yeast host cell culture in a batch or fed batch cultivated system using high density culture, wherein the media is supplemented with additives, such as glucose, glycerol, either at the time of inoculation or later at the time when the cells are grown to a high cell density, wherein high cell density is a cell density where the transformed yeast cells are grown to a density of up to 60 at OD600 and up to 120-300 g/L, WCW in a time interval of 12-24 hours at a growth temperature of 28-32° C.;
ii. growing the transformed yeast cell in culture to a stage of log phase growth;
iii. cooling the cultivation media to a temperature in range of 25-28° C.;
iv. inducing the cultivation media with induction agents;
v. harvesting the yeast cells containing the VLP by centrifugation or a microfiltration step, or a mixture of both;
vi. washing the harvested yeast cells with cell lysis buffer consisting of a diafiltration or buffer exchange step or an ultrafiltration step, or a mixture of both and resuspending the yeast host cells in the lysis buffer;
vii. disrupting the yeast host cells in lysis buffer by mechanical force or ultrasonic waves, or a French press or a method of cell lysis, or a combination of the above;
viii. purifying the VLPs obtained in step (vi), using an ion exchange resin or a mix mode resin or a combination of the two wherein said exchange resin is an anion or cation or mix mode resin, Capto Core 70;
ix. eluting the VLPs with a buffer containing up to 1-2 M KCl;
x. dialysing the VLPs with a formulation buffer comprising 20-100 mM Potassium phosphate pH 7.2.
xi. supplementing the VLPs with 50-100 mM KCl, optionally along with 0.0005-0.001% Tween 80 and 2-10% Sucrose.
xii. filter-sterilizing the dialyzed VLPs using 0.2 μm filter;
xiii. determining the yield of purified VLP.

40. The method as claimed in claim 39, wherein the high-density culture of yeast cells and cultivation medium is supplemented with an amount of an induction agent that comprises galactose, glycerol or a mixture of both, and wherein the period of induction is from 48-120 hours at 25-28° C.

41. The method as claimed in claim 39, wherein the cultivation medium is further supplemented with a boosting solution containing supplements selected from peptides, amino acids, tryptone and yeast extract.

42. The method as claimed in claim 39, wherein the cultivation media is saturated by air to a level 20-80% saturation.

43. The method as claimed in claim 39, wherein the lysis buffer of step (vi) contains 20 mM-100 mM Potassium phosphate pH 7.2, 0.0005%-0.001% Tween 80 or Tween 20 or a non-ionic detergent, 2 mM PMSF and wherein the lysis buffer is optionally supplemented with a nuclease.

44. The method as claimed in claim 39, wherein the yeast cells containing the VLP in lysis buffer is disrupted by mechanical force with a high pressure ultrasonic waves, or a method of cell lysis, where in the yeast cells are lysed or broken to obtain a lysed medium of 60-100% lysed cells in the lysis buffer.

45. The method as claimed in claim 39, wherein VLPs are harvested by a centrifugation step, or a diafiltration or an ultrafiltration step or a microfiltration step, or a mixture of both, to produce partially isolated VLPs.

46. The method as claimed in claim 39, wherein the eluted VLPs obtained from step (x), are dialysed by buffer exchange with a formulation buffer, consisting of 20-100 mM Potassium phosphate pH 7.2.

47. An immunogenic composition, comprising the VLPs as claimed in claim 32, along with pharmaceutically acceptable excipients, adjuvants and/or stabilizers.

48. A method of administering the composition as claimed in claim 47, wherein said composition is administered intranasal, mucosal, intradermally, subcutaneously, intramuscularly, sublingual or orally.

49. A vaccine comprising the VLPs as claimed in claim 32, wherein said vaccine induces an immune response in a subject.

50. The immunogenic composition as claimed in claim 47, wherein said immunogenic composition induces an immune response in a subject against multiple serotypes or clades of SARS-CoV virus.

51. The immunogenic composition as claimed in claim 47, wherein the said SARS-CoV virus is SARS-CoV2 virus.

52. The immunogenic composition as claimed in claim 47, wherein the subject is a human or non-human animal.
